(19) United States
(12) Patent Application Publication
(10) Pub. No.: US 2025/0312600 A1
(43) Pub. Date: Oct. 9, 2025

(54) NEUROMODULATION DEVICES AND ASSOCIATED SYSTEMS AND METHODS

(71) Applicant: XII Medical, Inc., Union City, CA (US)

(72) Inventors: Anthony V. Caparso, North Ridgeville, OH (US); Andrew E. Wu, Los Altos Hills, CA (US); Rabih Nassif, Santa Ana, CA (US); Nicholas Z. Pachon, San Francisco, CA (US); John P. Sam, Los Altos, CA (US); Paramjit S. Banwait, Fremont, CA (US)

(21) Appl. No.: 19/094,244

(22) Filed: Mar. 28, 2025

Related U.S. Application Data
(60) Provisional application No. 63/573,726, filed on Apr. 3, 2024.

(51) Int. Cl. A61N 1/36; A61N 1/05; A61N 1/378

(52) U.S. Cl. CPC A61N 1/3606; A61N 1/0526; A61N 1/3787

(57) ABSTRACT

Neuromodulation devices and associated systems and methods are disclosed herein. Various embodiments of the present technology relate to devices, systems, and methods for delivering electrical energy to a hypoglossal nerve of a patient. According to some embodiments, the present technology includes an implantable device comprising a lead and an antenna. The antenna can comprise a planar coil including a conductive wire formed into a wound portion with a plurality of spiral turns, and wherein the coil has a coil width measured in a first dimension and a coil length smaller than the coil width measured in a second dimension. The conductive wire can be biocompatible and non-corrosive.

### CROSS-REFERENCE TO RELATED APPLICATION(S)

The present application claims the benefit of priority to 63/573,726, filed Apr. 3, 2024, which is incorporated by reference herein in its entirety.

The present application is related to the following applications, each of which is incorporated by reference herein in its entirety: U.S. patent application Ser. No. 16/865,541, filed May 4, 2020, titled IMPLANTABLE STIMULATION POWER RECEIVER, SYSTEMS, AND METHODS, U.S. patent application Ser. No. 16/866,488, filed May 4, 2020, titled SYSTEMS AND METHODS TO IMPROVE SLEEP DISORDERED BREATHING USING CLOSED-LOOP FEEDBACK, U.S. patent application Ser. No. 16/866,523, filed May 4, 2020, titled SYSTEMS AND METHODS FOR IMPROVING SLEEP DISORDERED BREATHING, U.S. patent application Ser. No. 16/865,668, filed May 4, 2020, titled BIASED NEUROMODULATION LEAD AND METHOD OF USING SAME, and U.S. patent application Ser. No. 18/475,818, filed Sep. 27, 2023, titled NEUROMODULATION DEVICES AND ASSOCIATED SYSTEMS AND METHODS.

### TECHNICAL FIELD

The present technology relates to neuromodulation devices and associated systems and methods. Various embodiments of the present technology relate to neuromodulation devices, systems, and methods for treating sleep disordered breathing.

### BACKGROUND

Sleep disordered breathing (SDB), such as upper airway sleep disorders (UASDs), is a condition that occurs that diminishes sleep time and sleep quality, resulting in patients exhibiting symptoms that include daytime sleepiness, tiredness, and lack of concentration. Obstructive sleep apnea (OSA), the most common type of SDB, affects one in five adults in the United States. One in 15 adults has moderate to severe OSA and requires treatment. Untreated OSA results in reduced quality of life measures and increased risk of disease, including hypertension, stroke, heart disease, and others.

OSA is characterized by the complete obstruction of the airway, causing breathing to cease completely (apnea) or partially (hypopnea). During sleep, the tongue muscles relax. In this relaxed state, the tongue may lack sufficient muscle tone to prevent the tongue from changing its normal tonic shape and position. When the base of the tongue and/or soft tissue of the upper airway collapse, the upper airway channel is blocked, causing an apnea event. Blockage of the upper airway prevents air from flowing into the lungs, thereby decreasing the patient's blood oxygen level, which in turn increases blood pressure and heart dilation. This causes a reflexive forced opening of the upper airway channel until normal patency is regained, followed by normal respiration until the next apneic event. These reflexive forced openings briefly arouse the patient from sleep.

Current treatment options range from drug intervention, non-invasive approaches, to more invasive surgical procedures. In many of these instances, patient acceptance and therapy compliance are well below desired levels, rendering the current solutions ineffective as a long-term solution. Continuous positive airway pressure (CPAP), for example, is a standard treatment for OSA. While CPAP is non-invasive and highly effective, it is not well tolerated by all patients and has several side effects. Patient compliance and/or tolerance for CPAP is often reported to be between 40% and 60%. Surgical treatment options for OSA, such as anterior tongue muscle repositioning, orthognathic bimaxillary advancement, uvulopalatopharyngoplasty, and tracheostomy are available too. However, these procedures tend to be highly invasive, irreversible, and have poor and/or inconsistent efficacy. Even the more effective surgical procedures are undesirable because they usually require multiple invasive and irreversible operations, they may alter a patient's appearance (e.g., maxillo-mandibular advancement), and/or they may be socially stigmatic (e.g., tracheostomy) and have extensive morbidity.

### SUMMARY

The subject technology is illustrated, for example, according to various aspects described below, including with reference to FIGS. 1A-19. Various examples of aspects of the subject technology are described as numbered clauses (1, 2, 3, etc.) for convenience. These are provided as examples and do not limit the subject technology.

1. An implantable device comprising:
a lead comprising a proximal portion and a distal portion opposite the proximal portion along a longitudinal dimension of the lead, the distal portion comprising a first arm carrying a first electrode and a second arm carrying a second electrode, wherein the lead is configured to be implanted in a patient's body with the first arm positioned proximate a left hypoglossal nerve of the patient and the second arm positioned proximate a right hypoglossal nerve of the patient; and
an antenna positioned at the proximal portion of the lead, the antenna being configured to be positioned proximate a mylohyoid of the patient and configured to induce a current when positioned within an alternating magnetic field for supplying electrical energy to at least one of the first electrode or the second electrode, the antenna comprising a planar coil including a conductive wire formed into a wound portion with a plurality of spiral turns, and wherein the coil has a coil width measured in a first dimension and a coil length smaller than the coil width measured in a second dimension.

2. The device of Clause 1, wherein a distance between adjacent turns of the plurality of spiral turns varies along the coil width.

3. The device of Clause 1 or Clause 2, wherein each turn comprises a straight portion and a curved portion, and wherein a first pitch of the coil between straight portions of adjacent spiral turns is substantially constant, and wherein a second pitch of the coil between curved portions of adjacent spiral turns is greater than the first pitch.

4. The device of any one of Clauses 1-3, wherein the conductive wire is biocompatible and non-corrosive.

5. The device of any one of Clauses 1-4, wherein the conductive wire comprises at least one of gold, graphene, platinum, titanium, or an alloy thereof.

6. The device of any one of Clauses 1-5, wherein each turn extends from a first end to a second end, and wherein first ends of the plurality of spiral turns are aligned with one another along the coil width and second ends of the plurality of spiral turns are aligned with one another along the coil width.

7. The device of any one of Clauses 1-6, wherein each turn extends from a first end to a second end, and wherein a first end of one of the turns is continuous with a second end of a preceding one of the turns.

8. The device of Clause 6 or Clause 7, wherein the wound portion defines a plane and the first and second ends of each turn lie within the plane.

9. The device of any one of Clauses 1-8, wherein the antenna is configured to conform to the mylohyoid of the patient once implanted.

10. The device of any one of Clauses 1-9, wherein the antenna is flexible.

11. The device of any one of Clauses 1-10, wherein the antenna comprises a substrate carrying the coil.

12. The device of Clause 11, wherein the substrate defines a plurality of grooves configured to retain the plurality of spiral turns.

13. The device of Clause 11 or Clause 12, wherein the substrate is flexible.

14. The device of any one of Clauses 11-13, wherein the substrate is hydrophobic.

15. The device of any one of Clauses 11-14, wherein the substrate is biocompatible.

16. The device of any one of Clauses 11-15, wherein the substrate comprises a urethane or a silicone.

17. The device of any one of Clauses 11-16, wherein the substrate comprises an elongate shaft with a lumen extending therethrough, the elongate shaft being formed into a shaft wound portion with a plurality of shaft spiral turns, and wherein the conductive wire is disposed within the lumen of the elongate shaft.

18. The device of any one of Clauses 1-17, wherein the antenna comprises a coating carried by the coil.

19. The device of Clause 18, wherein the coating is flexible.

20 The device of Clause 18 or Clause 19, wherein the coating is hydrophobic.

21. The device of any one of Clauses 18-20, wherein the coating is biocompatible.

22 The device of any one of Clauses 18-21, wherein the coating comprises a urethane or a silicone.

23. The device of any one of Clauses 18-22, wherein the coating does not hermetically seal the coil.

24. The device of any one of Clauses 1-23, wherein the first and second arms each extend distally and laterally from a proximal end at the proximal portion of the lead to a free distal end.

25. The device of any one of Clauses 1-24, wherein the coil length is no greater than about 23 mm.

26 The device of any one of Clauses 1-25, wherein, when the device is implanted, a maximum dimension of the device along a sagittal anatomical plane is no greater than about 23 mm.

27. The device of any one of Clauses 1-26, wherein the coil width is between about 40 mm and about 50 mm.

28. The device of any one of Clauses 1-27, wherein an innermost turn of the plurality of spiral turns defines an opening of the coil.

29 The device of Clause 28, wherein an anchor configured to secure to patient tissue is positioned within the coil opening.

30. The device of Clause 28 or Clause 29, wherein an electronics component is positioned within the coil opening.

31. The device of any one of Clauses 1-30, wherein the implantable device includes an electronics component arranged in a hermetic enclosure and electrically coupled to the antenna.

32. The device of Clause 31, wherein the enclosure defines a port for receiving an elongate member therethrough.

33. The device of Clause 31 or Clause 32, wherein a first end portion of the conductive wire is configured to extend through a first port in the enclosure and a second end portion of the conductive wire is configured to extend through a second port in the enclosure.

34 The device of any one of Clauses 31-33, wherein the enclosure defines a port for receiving multiple elongate members therethrough.

35. The device of any one of Clauses 31-34, wherein the enclosure comprises a coating.

36 The device of Clause 35, wherein the coating comprises an epoxy.

37. The device of Clause 35 or Clause 36, wherein the coating comprises a polymer.

38. The device of any one of Clauses 35-37, wherein the coating comprises a first region comprising a parylene and a second region comprising a ceramic.

39 The device of any one of Clauses 35-38, wherein the coating comprises a plurality of first regions each comprising a parylene and a plurality of second regions each comprising a ceramic, wherein the first and second regions alternate along a thickness of the coating.

40. The device of any one of Clauses 1-39, wherein the planar coil is a first planar coil, the antenna further comprising a second planar coil including a second conductive wire formed into a second wound portion with a plurality of second spiral turns, wherein the second coil has a second coil width measured in the first dimension and a second coil length measured in the second dimension.

41. The device of Clause 40, wherein the first and second coils are spaced apart from one another along a thickness of the antenna.

42. The device of Clause 40 or Clause 41, wherein the plurality of spiral turns of the first coil is aligned with the plurality of second spiral turns of the second coil along the coil length and coil width of the first coil and the second coil length and second coil width of the second coil.

43. The device of any one of Clauses 40-42, wherein each turn of the first coil is individually electrically connected in parallel to a corresponding second turn of the second coil.

44 The device of Clause 43, wherein by virtue of each of the turns of the first coil being individually electrically connected in parallel to its respective corresponding second turn of the second coil, the antenna is configured to exhibit reduced parasitic capacitance when subjected to the alternating magnetic field.

45. The device of Clause 43 or Clause 44, wherein each turn of the first coil and its respective corresponding second turn of the second coil are electrically connected in parallel by at least one of laser welding, soldering, or tack welding.

46. The device of any one of Clauses 40-45, wherein the antenna comprises a substrate carrying the first and second coils.

47. The device of Clause 46, wherein the substrate has a first broad side and a second broad side opposite the first broad side along a thickness of the substrate, the first coil being positioned at the first broad side and the second coil being positioned at the second broad side.

48. The device of Clause 47, wherein the first broad side defines a plurality of first grooves configured to retain the plurality of spiral turns and the second broad side defines a plurality of second grooves configured to retain the plurality of second spiral turns.

49. The device of any one of Clauses 46-48, wherein each turn of the first coil is individually electrically connected in parallel to a corresponding second turn of the second coil via an electrical connector within the substrate.

50 The device of any one of Clauses 40-49, wherein the second conductive wire of the second coil is continuous with the conductive wire of the first coil.

51. An implantable neuromodulation lead comprising:
an extension portion having a proximal end portion configured to be coupled to an electronics component and a distal end portion; and
a lead body extending distally from the distal end portion of the extension portion, wherein the lead body branches into a first arm and a second arm and includes a first electrode disposed on the first arm and a second electrode disposed on the second arm,
wherein the lead body is configured to be implanted in a patient's body proximate a hypoglossal nerve and deliver an electrical signal to the hypoglossal nerve via the first and second electrodes.

52 The neuromodulation lead of Clause 51, wherein the lead body is configured to be implanted such that the first and second arms are aligned with and extend along a left hypoglossal nerve and a right hypoglossal nerve, respectively.

53. The neuromodulation lead of any one of the preceding Clauses, wherein the first arm comprises a proximal region and a distal region, wherein the proximal region extends laterally away from the distal end portion of the extension portion and the distal region extends distally away from the proximal region, and wherein the first electrode is carried by the distal region.

54. The neuromodulation lead of any one of the preceding Clauses, wherein the distal region of the first arm extends distally away from the proximal region along a longitudinal dimension.

55. The neuromodulation lead of any one of the preceding Clauses, wherein the proximal region of the first arm is angled vertically away from the extension portion such that the distal region is positioned in a different plane than the extension portion.

56. The neuromodulation lead of any one of the preceding Clauses, wherein the second arm comprises a proximal region and a distal region, wherein the proximal region extends laterally away from the distal end portion of the extension portion and the distal region extends distally away from the proximal region, and wherein the second electrode is carried by the distal region.

57. The neuromodulation lead of any one of the preceding Clauses, wherein the distal region of the second arm extends distally away from the proximal region along a longitudinal dimension.

58. The neuromodulation lead of any one of the preceding Clauses, wherein the proximal region of the second arm is angled vertically away from the extension portion such that the distal region is positioned in a different plane than the extension portion.

59. The neuromodulation lead of any one of the preceding Clauses, wherein the proximal regions of the first arm and the second arm extend laterally away from the distal end portion of the extension portion in opposing directions.

60. The neuromodulation lead of any one of the preceding Clauses, further comprising a connector between the extension portion and the first and second arms, wherein the connector is coupled to the distal end portion of the extension portion, a proximal region of the first arm, and a proximal region of the second arm.

61. The neuromodulation lead of any one of the preceding Clauses, wherein the electrical signal is configured to treat sleep apnea.

62. An implantable neuromodulation lead comprising:
an extension portion having a proximal end portion configured to be coupled to an electronics component and a distal end portion; and
a lead body extending distally from the distal end portion of the extension portion, wherein the lead body branches into a left arm and a right arm and includes a left electrode disposed on the left arm and a right electrode disposed on the right arm, and wherein at least one of the left arm or the right arm is bent relative to the extension portion such that the at least one left or right arm is positioned at a different elevation than the extension portion.

63. The neuromodulation lead of any one of the preceding Clauses, wherein the lead body is configured to deliver electrical stimulation energy to a hypoglossal nerve of a patient to treat sleep disordered breathing.

64. The neuromodulation lead of any one of the preceding Clauses, wherein the right arm is configured to be positioned proximate a right hypoglossal nerve of a patient and the left arm is configured to be positioned proximate a left hypoglossal nerve of a patient.

65 The neuromodulation lead of any one of the preceding Clauses, wherein, when the lead is implanted, the at least one of the left arm or the right arm extends superiorly from a proximal end portion located at the extension portion and proximate a geniohyoid muscle of a patient to a distal end portion located proximate a genioglossus muscle of the patient.

66. The neuromodulation lead of any one of the preceding Clauses, wherein, when the lead is implanted, the proximal end portion of the extension portion is positioned inferior of a mylohyoid muscle of the patient and the distal end portion of the extension portion is positioned superior of a geniohyoid muscle of the patient.

67 The neuromodulation lead of any one of the preceding Clauses, wherein, when the lead is implanted, the extension portion is positioned at least partially between a right geniohyoid muscle and a left geniohyoid muscle of the patient.

68. An implantable neuromodulation lead comprising:
an extension portion having a proximal end portion configured to be coupled to an electronics component and a distal end portion; and
a lead body extending distally from the distal end portion of the extension portion, wherein the lead body branches into a left arm and a right arm and includes a left electrode disposed on the left arm and a right electrode disposed on the right arm,
wherein the lead body is configured to be implanted at least partially in a sublingual region of a patient and configured to deliver electrical stimulation energy to the sublingual region to treat sleep apnea.

69. The neuromodulation lead of any one of the preceding Clauses, wherein the lead body is configured to deliver electrical stimulation energy to the sublingual region to increase activity in tongue protrusor muscles of the patient.

70. The neuromodulation lead of any one of the preceding Clauses, wherein the lead body is configured to be implanted such that the left and right arms are at least partially positioned between a genioglossus muscle of the patient and a geniohyoid muscle of the patient.

71. The neuromodulation lead of any one of the preceding Clauses, wherein, when the lead is implanted, each of the left and right arms extends superiorly from a proximal end portion at the extension portion and proximate a geniohyoid muscle of a patient to a distal end portion proximate a genioglossus muscle of the patient.

72. The neuromodulation lead of any one of the preceding Clauses, wherein the right arm is configured to be positioned proximate a right hypoglossal nerve of a patient and the left arm is configured to be positioned proximate a left hypoglossal nerve of a patient.

73. The neuromodulation lead of any one of the preceding Clauses, wherein the lead body is configured to deliver electrical stimulation energy to a hypoglossal nerve of a patient to treat sleep apnea.

74. The neuromodulation lead of any one of the preceding Clauses, wherein, when the lead is implanted, the proximal end portion of the extension portion is positioned inferior of a mylohyoid muscle of the patient and the distal end portion of the extension portion is positioned superior of a geniohyoid muscle of the patient.

75. The neuromodulation lead of any one of the preceding Clauses, wherein, when the lead is implanted, the extension portion is positioned at least partially between a right geniohyoid muscle and a left geniohyoid muscle of the patient.

76. An implantable neuromodulation lead comprising:
a lead body comprising a left arm and a right arm joined at their proximal ends, wherein the left and right arms extend laterally away from one another, and wherein the lead body includes a left electrode disposed on the left arm and a right electrode disposed on the right arm,
wherein the lead body is configured to be implanted in a patient's body proximate a hypoglossal nerve for delivery of an electrical signal to the hypoglossal nerve via the left and right electrodes.

77. A neurostimulation lead for implanting at a treatment site within a patient, the neurostimulation lead comprising:
a lead body;
a plurality of electrodes carried by the lead body; and
a plurality of fixation members extending radially away from the lead body, wherein the fixation members are configured to anchor the lead body to tissue at the treatment site,
wherein the neurostimulation lead is configured to be implanted in a patient's body at the treatment site to delivery energy to the treatment site via the electrodes.

78. The neuromodulation lead of any one of the preceding Clauses, wherein the lead body comprises a polymer sidewall and the fixation members are cut from the polymer sidewall.

79. The neuromodulation lead of any one of the preceding Clauses, wherein the fixation members comprise first ends at the sidewall and second ends radially spaced apart from the sidewall.

80. The neuromodulation lead of any one of the preceding Clauses, wherein the fixation members extend no more than 0.5 mm away from the lead body.

81. The neuromodulation lead of any one of the preceding Clauses, wherein the fixation members are cantilevered from the sidewall.

82. The neuromodulation lead of any one of the preceding Clauses, wherein the polymer sidewall comprises a thermoplastic polyurethane.

83. The neuromodulation lead of any one of the preceding Clauses, wherein at least some of the fixation members are spaced apart along a length of the lead body.

84. The neuromodulation lead of any one of the preceding Clauses, wherein at least some of the fixation members are spaced apart around a circumference of the lead body.

85. The neuromodulation lead of any one of the preceding Clauses, wherein the lead is configured to deliver stimulation energy at the treatment site to treat sleep apnea.

86. The neuromodulation lead of any one of the preceding Clauses, wherein the lead body is configured to be positioned proximate a hypoglossal nerve of the patient.

87. The neuromodulation lead of any one of the preceding Clauses, wherein the lead body is configured to deliver stimulation energy to a hypoglossal nerve of the patient.

88. The neuromodulation lead of any one of the preceding Clauses, wherein the lead body is configured to detect activity of a lingual muscle and/or a suprahyoid muscle of the patient.

89. A neuromodulation lead comprising:
a lead body comprising a plurality of electrodes; and
an extension portion having a proximal end configured to be coupled to an electronic component and a distal end configured to be coupled to the lead body, the distal end being opposite the proximal end along a length of the extension portion, wherein the length of the extension portion is adjustable to vary a distance between the lead body and the electronic component,
wherein the lead is configured to be implanted in a patient's body at a treatment site to delivery energy to the treatment site via the electrodes.

90. The neuromodulation lead of any one of the preceding Clauses, wherein the extension portion is configured to bend along its longitudinal axis to vary the distance between the lead body and the electronic component.

91. The neuromodulation lead of any one of the preceding Clauses, wherein the extension portion comprises a helically wound portion.

92. The neuromodulation lead of any one of the preceding Clauses, wherein the extension portion comprises an undulating portion.

93. The neuromodulation lead of any one of the preceding Clauses, wherein the neurostimulation lead is configured to deliver stimulation energy at the treatment site to treat sleep apnea.

94. The neuromodulation lead of any one of the preceding Clauses, wherein the lead body is configured to be positioned proximate a hypoglossal nerve of the patient.

95. The neuromodulation lead of any one of the preceding Clauses, wherein the lead body is configured to deliver stimulation energy to a hypoglossal nerve of the patient via the electrodes.

96 The neuromodulation lead of any one of the preceding Clauses, wherein the lead body is configured to detect activity of a muscle of the patient.

97. An implantable antenna comprising:
a substrate comprising a substrate material; and
a coil arranged on the substrate and comprising a plurality of coil turns including a first coil turn and a second coil turn adjacent to the first coil turn;
wherein the substrate comprises at least one open region where the first coil turn is not joined to the second coil turn by substrate material.

98. The antenna of any one of the preceding Clauses, wherein the substrate comprises at least one strut region where the first coil turn is joined to the second coil turn by substrate material.

99. The antenna of any one of the preceding Clauses, wherein the at least one open region comprises an arcuate open region that extends along a partial circumference of the first coil turn.

100. The antenna of any one of the preceding Clauses, wherein the at least one open region comprises a plurality of arcuate open regions that each extends along a respective partial circumference of the first coil turn.

101. The antenna of any one of the preceding Clauses, wherein the partial circumference is at least about 50% of the circumference of the first coil turn.

102. The antenna of any one of the preceding Clauses, wherein the partial circumference is about 50% or less of the circumference of the first coil turn.

103. The antenna of any one of the preceding Clauses, wherein two or more adjacent coil turns are connected to each other by substrate material.

104. A neuromodulation lead, comprising the implantable antenna of any one of the preceding Clauses.

105. A method of treating sleep disordered breathing, comprising:
implanting a neuromodulation lead of any one of the preceding Clauses at a treatment site in a patient body; and
delivering stimulation energy to the treatment site via the electrodes of the neuromodulation lead.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a fragmentary midline sagittal view of an upper airway of a human patient.

FIG. 1B is an illustration of the musculature and hypoglossal innervation of the human tongue.

FIG. 1C is a schematic superior view of a distal arborization of right and left hypoglossal nerves of a human patient. The hypoglossal nerves of FIG. 1C are shown as extending anteriorly from the bottom of the page to the top of the page (e.g., from the hyoid bone to the anterior mandible).

FIG. 2A is a schematic illustration of a neuromodulation system configured in accordance with several embodiments of the present technology.

FIG. 2B is a perspective view of a neuromodulation device configured in accordance with several embodiments of the present technology.

FIGS. 2C and 2D are top and side views, respectively, of the neuromodulation device of FIG. 2B.

FIGS. 3A-3F are various views of the neuromodulation device shown in FIGS. 2B-2D implanted in a human patient in accordance with several embodiments of the present technology.

FIGS. 4A, 4B, and 4C are perspective, side, and end views, respectively, of a lead of the neuromodulation device shown in FIGS. 2B-2D.

FIG. 5 is a side view of a distal end portion of an arm of a lead of the neuromodulation device shown in FIGS. 2B-2D.

FIGS. 6A-6D are perspective, top, end, and side views, respectively, of a first connector of the neuromodulation device configured in accordance with several embodiments of the present technology.

FIGS. 7A-7C depict various configurations of an extension portion of a lead of a neuromodulation device configured in accordance with several embodiments of the present technology.

FIG. 8 illustrates a second connector of a neuromodulation device configured in accordance with several embodiments of the present technology.

FIG. 9 illustrates a second connector of a neuromodulation device in an open configuration and configured in accordance with several embodiments of the present technology.

FIGS. 10A-10C depict various configurations of electrical conductors within an extension portion of a lead of a neuromodulation device configured in accordance with several embodiments of the present technology.

FIG. 11 illustrates a neuromodulation device configured in accordance with several embodiments of the present technology.

FIGS. 12A-12H illustrate various configurations of an antenna in a neuromodulation device configured in accordance with several embodiments of the present technology.

FIG. 13 is a plan view of an antenna of a neuromodulation device configured in accordance with several embodiments of the present technology.

FIG. 14 is a plan view of an antenna of a neuromodulation device configured in accordance with several embodiments of the present technology.

FIG. 15 is an exploded perspective view of an antenna of a neuromodulation device configured in accordance with several embodiments of the present technology.

FIGS. 16 and 17 are cross-sectional views of substrates of antennas of neuromodulation devices configured in accordance with several embodiments of the present technology.

FIG. 18 is a perspective view of a substrate of an antenna of a neuromodulation device configured in accordance with several embodiments of the present technology.

FIG. 19 is a side schematic view of a neuromodulation device configured in accordance with several embodiments of the present technology.

### DETAILED DESCRIPTION

The present disclosure relates to neuromodulation systems, which can be used to provide a variety of electrical therapies, including neuromodulation therapies such as nerve and/or muscle stimulation. Stimulation can induce excitatory or inhibitory neural or muscular activity. Such therapies can be used at various suitable sites within a patient's anatomy. According to some embodiments, the neuromodulation systems of the present technology are configured to treat sleep disordered breathing (SDB), including obstructive sleep apnea (OSA) and/or mixed sleep apnea, via neuromodulation of the hypoglossal nerve (HGN).

For the purpose of contextualizing the structure and operation of the neuromodulation systems and devices disclosed herein, some of the relevant anatomy and physiology are first described below. The headings provided herein are for convenience only and do not interpret the scope or meaning of the claimed present technology. Embodiments under any one heading may be used in conjunction with embodiments under any other heading. For example, any of the neuromodulation systems and devices described in connection with Section II can include any of the neuromodulation devices described in connection with Section III.

### I. Anatomy and Physiology

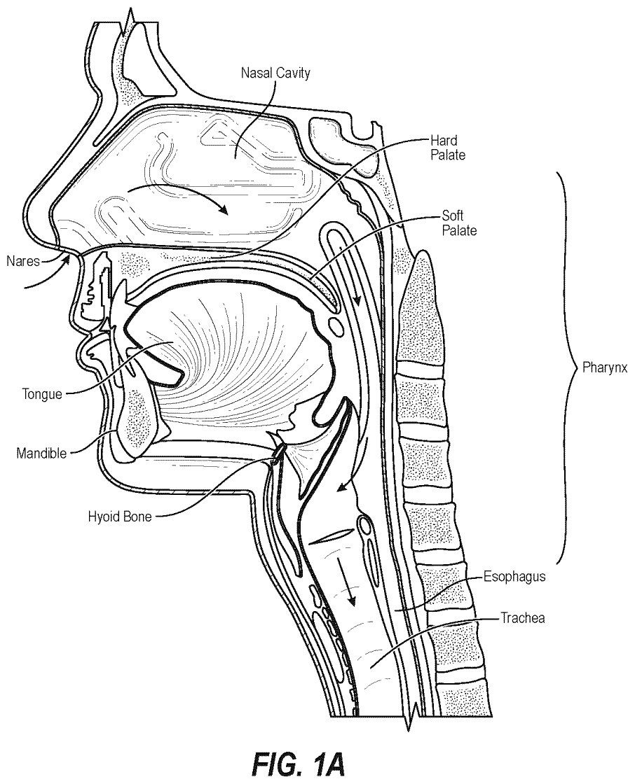

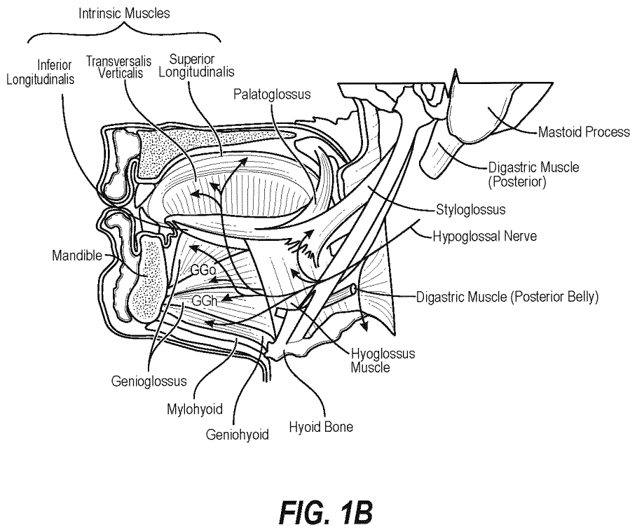

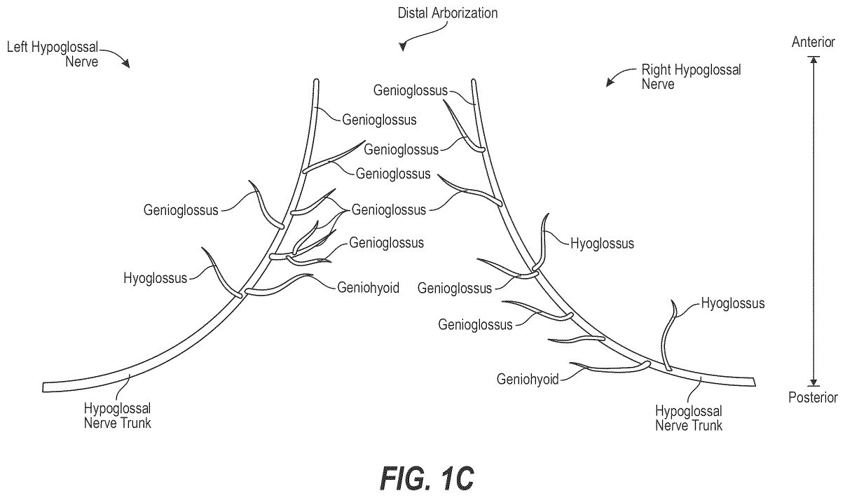

As previously mentioned, respiration in patients with SDB is frustrated due to obstruction, narrowing, and/or collapse of the upper airway during sleep. As shown in FIG. 1A, the upper airway comprises the nasal cavity, the oral cavity, the pharynx, and the larynx. Patency of the upper airway and resistance to airflow in the upper airway are controlled by a complex network of muscles under both voluntary and involuntary neuromuscular control. For example, the muscles of the tongue, the suprahyoid muscles (e.g., the geniohyoid, mylohyoid, stylohyoid, hyoglossus, and the anterior belly of the digastric muscle), and the muscles comprising the soft palate (e.g., palatal muscles) open, widen, and/or stabilize the upper airway during inspiration to counteract the negative airway pressure responsible for drawing air into the airway and the lungs.

With reference to FIG. 1B, the tongue comprises both intrinsic and extrinsic lingual muscles. Generally, activation of the intrinsic muscles changes the shape of the tongue while activation of the extrinsic muscles tends to move the position of the whole tongue. The extrinsic muscles originate at a bony attachment and insert within the tongue. They comprise the genioglossus muscle, the styloglossus muscle, the hyoglossus muscle, and the palatoglossus muscle. The intrinsic muscles both originate and insert within the tongue, and comprise the superior longitudinalis, the inferior longitudinalis, the transversalis, and the verticalis. In a patient who is awake, the brain supplies neural drive to these muscles through the HGN to maintain tongue shape and position, preventing the tongue from blocking the airway.

The lingual muscles are also functionally categorized as either retrusor or protrusor muscles and both intrinsic and extrinsic muscles fall into these categories. The retrusor muscles include the intrinsic superior and inferior longitudinalis muscles and the extrinsic hyoglossus and styloglossus muscles. The protrusor muscles include the intrinsic verticalis and transversalis muscles and the extrinsic genioglossus muscle. Contraction of the styloglossus muscle causes elevation of the tongue while depression of the tongue is the result of downward movements of hyoglossus and genioglossus muscles. Also labeled in FIG. 1B is the geniohyoid muscle, which is a suprahyoid muscle (not a tongue muscle) but still an important protrusor and pharyngeal dilator, and thus contributes to maintaining upper airway patency. It is believed that effective treatment of OSA requires stimulation of the protrusor muscles with minimal or no activation of the retrusor muscles. Thus, for neuromodulation therapy to be effective it is considered beneficial to localize stimulation to the protrusor muscles while avoiding activation of the retrusor muscles.

The largest of the tongue muscles, the genioglossus, comprises two morphological and functional compartments according to fiber distribution, action, and nerve supply. The first, the oblique compartment (GGo), includes vertical fibers that, when contracted, depress the tongue without substantially affecting pharyngeal patency. The second, the horizontal compartment (GGh), contains longitudinal fibers that, when activated, protrude the posterior part of the tongue and enlarge the pharyngeal opening. The GGo contains Type II muscle fibers that are quickly fatigued, whereas the GGh contains Type I muscle fibers that are slower to fatigue. Accordingly, it can be advantageous to stimulate the GGh with little or no stimulation of the GGo to effectively protrude the tongue while preventing or limiting fatigue of the tongue.

The suprahyoid muscles, which comprise the mylohyoid, the geniohyoid, the stylohyoid, and the digastric (only a portion of which is shown in FIG. 1B), extend between the mandible and the hyoid bone to form the floor of the mouth. The geniohyoid is situated inferior to the genioglossus muscle of the tongue and the mylohyoid is situated inferior to the geniohyoid. Contraction of the geniohyoid and tone of the sternohyoid (an infrahyoid muscle, not shown) cooperate to pull the hyoid bone anteriorly to open and/or widen the pharyngeal lumen and stabilize the anterior wall of the hypopharyngeal region. In contrast to the genioglossus and geniohyoid, which are considered tongue protrusors, the hyoglossus and styloglossus are considered tongue retrusors. Activation of the hyoglossus and styloglossus tends to retract the tongue posteriorly, which reduces the size of the pharyngeal opening, increases airway resistance, and frustrates respiration.

As previously mentioned, all of the extrinsic and intrinsic muscles of the tongue are innervated by the HGN, with the exception of the palatoglossus, which is innervated by the vagal nerve. There are two hypoglossal nerves in the body, one on the right side of the head and one on the left side. Each hypoglossal nerve originates at a hypoglossal nucleus in the medulla oblongata of the brainstem, exits the cranium via the hypoglossal canal, and passes inferiorly through the retrostyloid space (a portion of the lateral pharyngeal space) to the occipital artery. The hypoglossal nerve then curves and courses anteriorly to the muscles of the tongue, passing between the anterior edge of the hyoglossus muscle and the posterior edge of the mylohyoid muscle into the sublingual area where it splits into its distal arborization.

FIG. 1C is a schematic superior view of the distal arborization of the right and left hypoglossal nerves. Referring to FIGS. 1B and 1C together, the HGN comprises (1) portions of the distal arborization that innervate the styloglossus and the hyoglossus (tongue retrusor muscles) and (2) portions of the distal arborization that innervate the intrinsic muscles of the tongue, the genioglossus, and the geniohyoid (tongue protrusor muscles). Additionally, the portions of the distal arborization that innervate the tongue retrusor muscles tend to be located posterior of the portions of the distal arborization that innervate the tongue protrusor muscles.

A reduction in activity of the muscles responsible for airway maintenance can result in an increase in airway resistance and a myriad of downstream effects on a patient's respiration and health. Activity of the genioglossus muscle, for example, can decrease during sleep which, whether alone or in combination with other factors (e.g., airway length, airway diameter, soft tissue volume, premature wakening, etc.), can result in substantial airway resistance and/or airway collapse leading to sleep disordered breathing, such as OSA. It is believed that in order for neuromodulation therapy to be effective, it may be beneficial to largely confine stimulation of the HGN to the portions of the distal arborization that innervate protrusor muscles while avoiding or limiting stimulation of the portions of the distal arborization that activate the retrusor muscles.

### II. Neuromodulation Systems

Various embodiments of the present technology are directed to devices, systems, and methods for modulating neurological activity and/or control of one or more nerves associated with one or more muscles involved in airway maintenance. Such neuromodulation can increase activity in targeted muscles, for example the genioglossus and geniohyoid, to reduce a patient's airway resistance and improve the patient's respiration. Moreover, targeted modulation of specific portions of the distal arborization of the hypoglossal nerve can increase activity in tongue protrusor muscles without substantially increasing activity in tongue retrusor muscles to provide a highly efficacious treatment. Additionally or alternatively, targeted modulation of specific portions of the distal arborization of the hypoglossal nerve that innervate the GGh but not portions of the distal arborization of the hypoglossal nerve that innervate the GGo can be used to effectively protrude the tongue while preventing or limiting fatigue of the tongue.

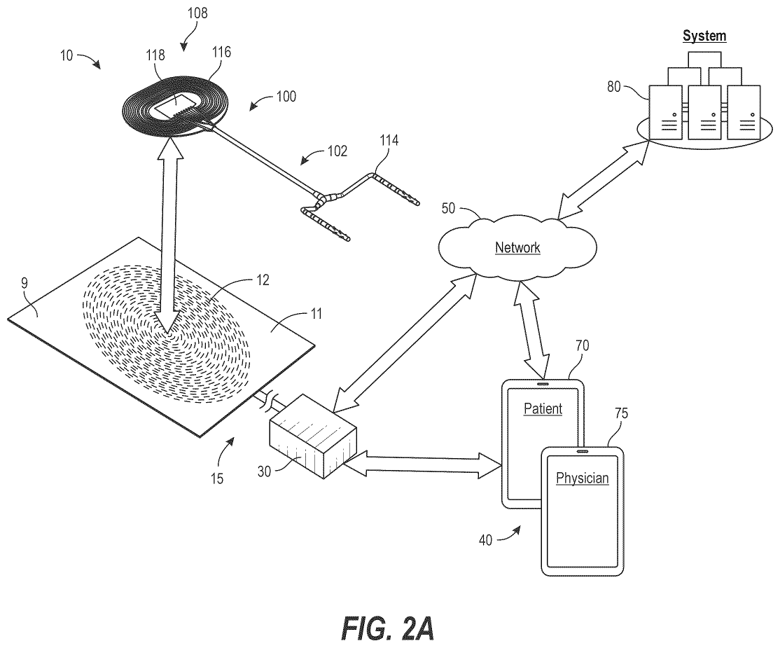

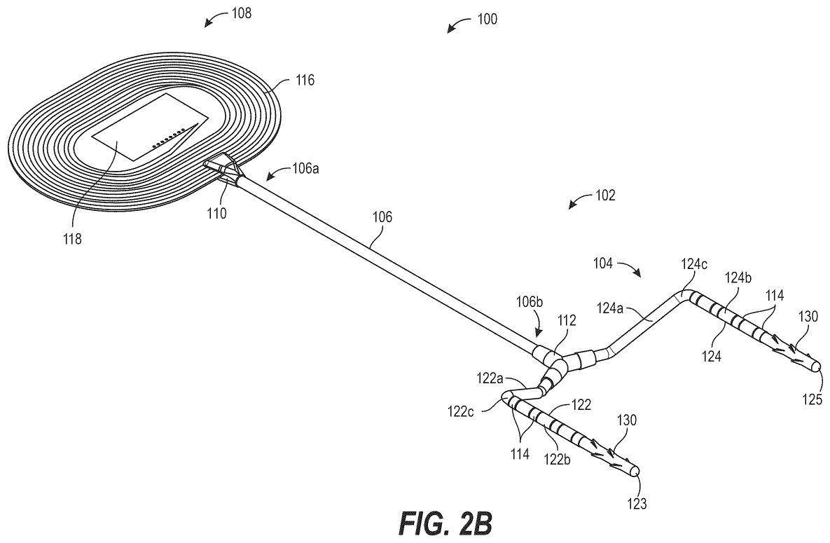

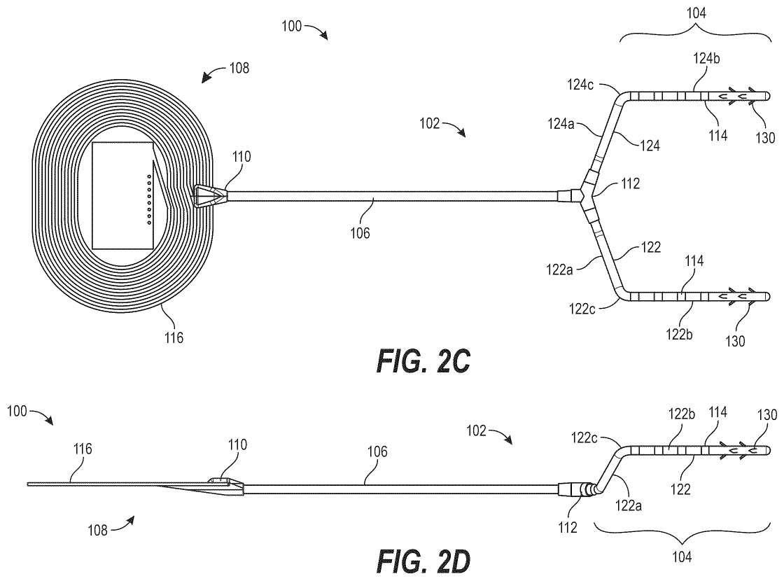

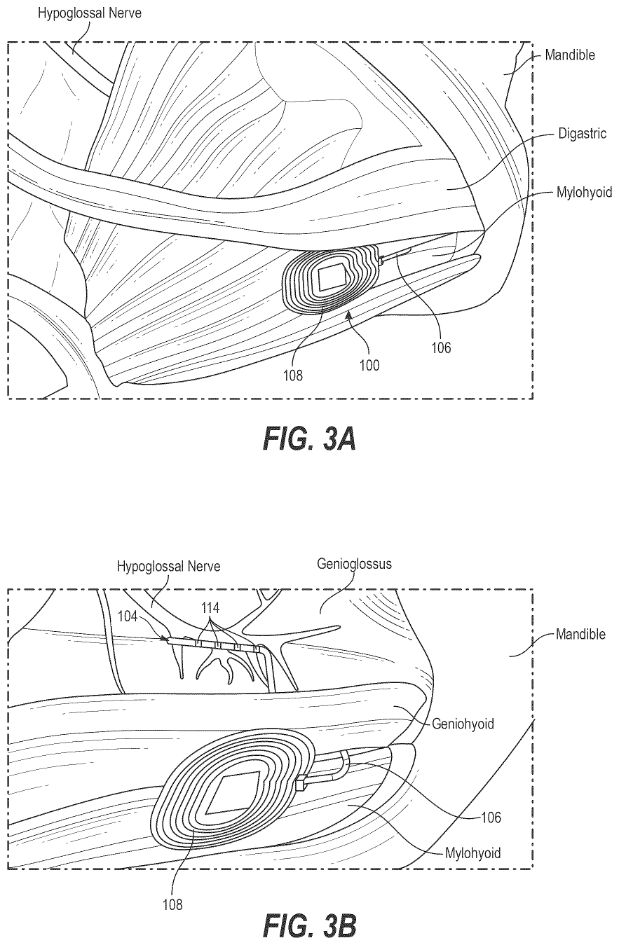

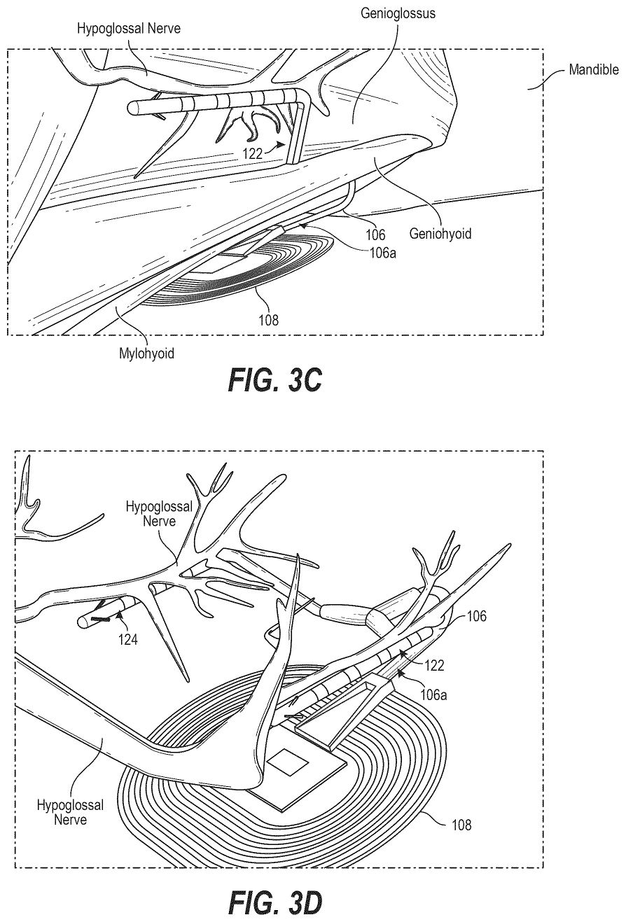

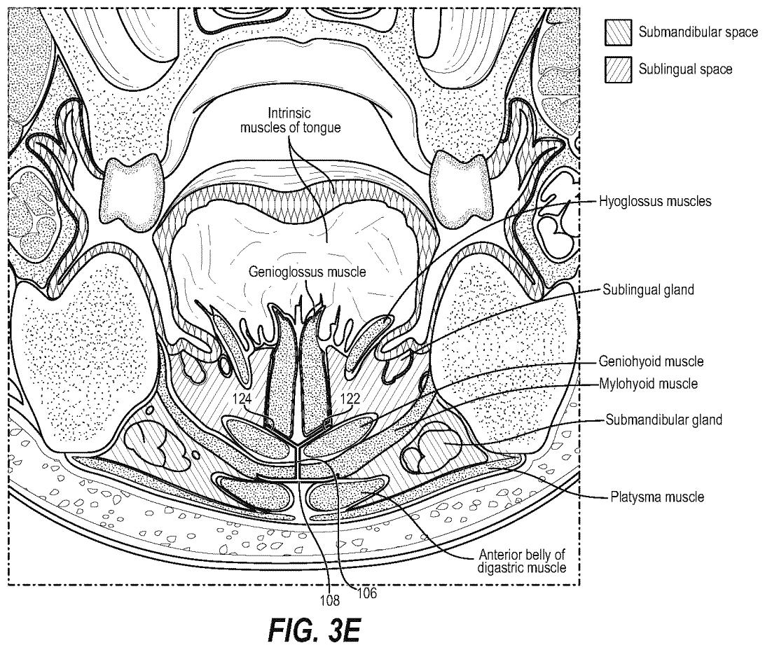

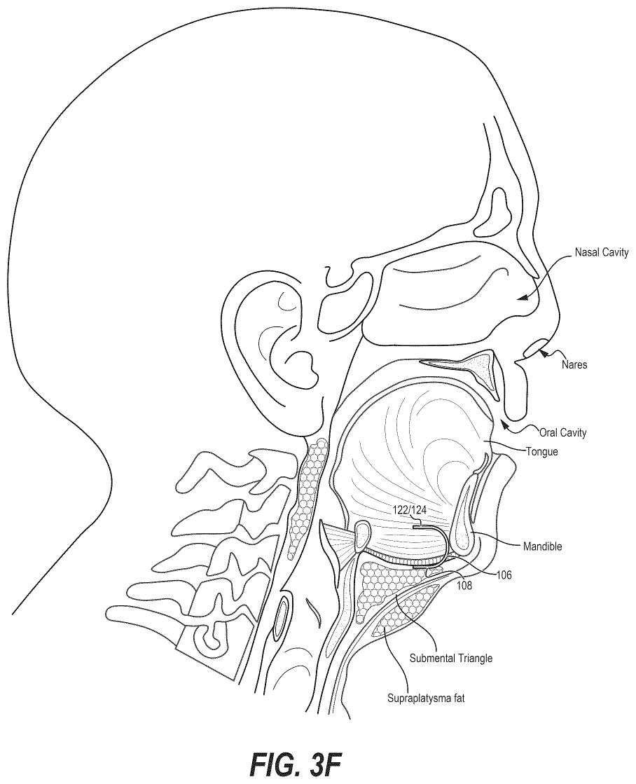

FIG. 2A shows a neuromodulation system 10 for treating SDB configured in accordance with the present technology. The system 10 can include an implantable neuromodulation device 100 and an external system 15 configured wirelessly couple to the neuromodulation device 100. The neuromodulation device 100 can include a lead 102 having a plurality of conductive elements 114 and an electronics package 108 having a first antenna 116 and an electronics component 118. The neuromodulation device 100 is configured to be implanted at a treatment site comprising submental and sublingual regions of a patient's head, as detailed below with reference to FIGS. 3A-3F.

In use, the electronics package 108 or one or more elements thereof can be configured provide a stimulation energy to the conductive elements 114 that has a pulse width, amplitude, duration, frequency, duty cycle, and/or polarity such that the conductive elements 114 apply an electric field at the treatment site that modulates the hypoglossal nerve. The stimulation energy can be delivered according to a periodic waveform including, for example, a charge-balanced square wave comprising alternating anodic and cathodic pulses.

One or more pulses of the stimulation energy can have a pulse width between about 10 μs and about 1000 μs, between about 50 μs and about 950 μs, between about 100 μs and about 900 μs, between about 150 μs and about 800 μs, between about 200 μs and about 850 μs, between about 250 μs and about 800 μs, between about 300 μs and about 750 μs, between about 350 μs and about 700 μs, between about 400 μs and about 650 μs, between about 450 μs and about 600 μs, between about 500 μs and about 550 μs, about 50 μs, about 100 μs, about 150 μs, about 200 μs, about 250 μs, about 300 μs, about 350 μs, about 400 μs, about 450 μs, about 500 μs, about 550 μs, about 600 μs, about 650 μs, about 700 μs, about 750 μs, about 800 μs, about 850 μs, about 900 μs, about 950 μs, and/or about 1000 μs. In some embodiments, one or more pulses of the stimulation energy has a pulse width of between about 50 μs and about 450 μs.

One or more pulses of the stimulation energy can have an amplitude sufficient to cause an increase in phasic activity of a desired muscle. For example, one or more pulses of the stimulation energy can have a current-controlled amplitude between about 0.1 mA and about 5 mA. In some embodiments, the stimulation energy has an amplitude of about 0.3 mA, about 0.4 mA, about 0.5 mA, about 0.6 mA, about 0.7 mA, about 0.8 mA, about 0.9 mA, about 1 mA, about 1.5 mA, about 2 mA, about 2.5 mA, about 3 mA, about 3.5 mA, about 4 mA, about 4.5 mA, and/or about 5 mA. Additionally or alternatively, an amplitude of one or more pulses of the stimulation energy can be voltage-controlled. An amplitude of one or more pulses of the stimulation energy can be based at least in part on a size and/or configuration of the conductive elements 114, a location of the conductive elements 114 in the patient, etc.

A frequency of the pulses of the stimulation energy can be between about 10 Hz and about 50 Hz, between about 20 Hz and about 40 Hz, about 10 Hz, about 15 Hz, about 20 Hz, about 25 Hz, about 30 Hz, about 35 Hz, about 40 Hz, about 45 Hz, and/or about 50 Hz. In some embodiments, the frequency can be based on a desired effect of the stimulation energy on one or more muscles or nerves. For example, lower frequencies may induce a muscular twitch whereas higher frequencies may include complete contraction of a muscle.

The external system 15 can comprise an external device 11 and a control unit 30 communicatively coupled to the external device 11. In some embodiments, the external device 11 is configured to be positioned proximate a patient's head while they sleep. The external device 11 can comprise a carrier 9 integrated with a second antenna 12. While the control unit 30 is shown separate from the external device 11 in FIG. 2A, in some embodiments the control unit 30 can be integrated with and/or a portion of the external device 11. The second antenna 12 can be configured for multiple purposes. For example, the second antenna 12 can be configured to power the neuromodulation device 100 through electromagnetic induction. Electrical current can be induced in the first antenna 116 when it is positioned above the second antenna 12 of the external device 11, in an electromagnetic field produced by second antenna 12. The first and second antennas 116, 12 can also be configured transmit data to and/or receive data from one another via one or more wireless communication techniques (e.g., Bluetooth, WiFi, USB, etc.) to facilitate communication between the neuromodulation device 100 and the external system 15. This communication can, for example, include programming, e.g., uploading software/firmware revisions to the neuromodulation device 100, changing/adjusting stimulation settings and/or parameters, and/or adjusting parameters of control algorithms.

The control unit 30 of the external system 15 can include a processor and/or a memory that stores instructions (e.g., in the form of software, code or program instructions executable by the processor or controller) for causing the external device to generate an electromagnetic field according to certain parameters provided by the instructions. The external system can include and/or be configured to be coupled to a power source such as a direct current (DC) power supply, an alternating current (AC) power supply, and/or a power supply switchable between DC and AC. The processor of the external system can be used to control various parameters of the energy output by the power source, such as intensity, amplitude, duration, frequency, duty cycle, and polarity. Instead of or in addition to a processor, the external system can include drive circuitry. In such embodiments, the external system can include hardwired circuit elements to provide the desired waveform delivery rather than a software-based generator. The drive circuitry can include, for example, analog circuit elements (e.g., resistors, diodes, switches, etc.) that are configured to cause the power source to supply energy to the second antenna 12 to produce an electromagnetic field according to the desired parameters. In some embodiments, the neuromodulation device 100 can be configured for communication with the external system via inductive coupling.

The system 10 can also include a user interface 40 in the form of a patient device 70 and/or a physician device 75. The user interface(s) 40 can be configured to transmit and/or receive data with the external system 15, the second antenna 12, the control unit 30, the neuromodulation device 100, and/or the remote computing device(s) 80 via wired and/or wireless communication techniques (e.g., Bluetooth, WiFi, USB, etc.). In the example configuration of FIG. 2A, both the patient device 70 and physician device 75 are smartphones. The type of device could, however, vary. One or both of the patient device 70 and physician device 75 can have an application or “app” installed thereon that is user specific, e.g., a patient app or a physician app, respectively. The patient app can allow the patient to execute certain commands necessary for controlling operation of neuromodulation device 100, such as, for example, start/stop therapy, increase/decrease stimulation power or intensity, and/or select a stimulation program. In addition to the controls afforded the patient, the physician app can allow the physician to modify stimulation settings, such as pulse settings (patterns, duration, waveforms, etc.), stimulation frequency, amplitude settings, and electrode configurations, closed-loop and open loop control settings and tuning parameters for the embedded software that controls therapy delivery during use.

The patient and/or physician devices 70, 75 can be configured to communicate with the other components of the system 10 via a network 50. The network 50 can be or include one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof. The patient and/or physician devices 70, 75 can be configured to communicate with one or more remote computing devices 80 via the network 50 to enable the transfer of data between the devices 70, 75 and the remote computing device(s) 80. Additionally, the external system 15 can be configured to communicate with the other components of the system 10 via the network 50. This can also enable the transfer of data between the external system 15 and remote computing device(s) 80.

The external system 15 can receive the programming, software/firmware, and settings/parameters through any of the communication paths described above, e.g., from the user interface(s) 40 directly (wired or wirelessly) and/or through the network 50. The communication paths can also be used to download data from the neuromodulation device 100, such as measured data regarding completed stimulation therapy sessions, to the external system 15. The external system 15 can transmit the downloaded data to the user interface 40, which can send/upload the data to the remote computing device(s) 80 via the network 50.

In addition to facilitating local control of the system 10, e.g., the external system 15 and the neuromodulation device 100, the various communication paths shown in FIG. 2A can also enable:

Distributing from the remote computing device(s) 80 software/firmware updates for the patient device 70, physician device 75, external system 15, and/or neuromodulation device 100.

Downloading from the remote computing device(s) 80 therapy settings/parameters to be implemented by the patient device 70, physician device 75, external system 15, and/or neuromodulation device 100.

Facilitating therapy setting/parameter adjustments/algorithm adjustments by a remotely located physician.

Uploading data recorded during therapy sessions.

Maintaining coherency in the settings/parameters by distributing changes and adjustments throughout the system components.

The therapeutic approach implemented with the system 10 can involve implanting only the neuromodulation device 100 and leaving the external system 15 as an external component to be used only during the application of therapy. To facilitate this, the neuromodulation device 100 can be configured to be powered by the external system 15 through electromagnetic induction. In operation, the second antenna 12, operated by control unit 30, can be positioned external to the patient in the vicinity of the neuromodulation device 100 such that the second antenna 12 is close to the first antenna 116 of the neuromodulation device 100. In some embodiments, the second antenna 12 is carried by a flexible carrier 9 that is configured to be positioned on or sufficiently near the sleeping surface while the patient sleeps to maintain the position of the first antenna 116 within the target volume of the electromagnetic field generated by the second antenna 12. Through this approach, the system 10 can deliver therapy to improve SDB (such as OSA), for example, by stimulating the HGN through a shorter, less invasive procedure. The elimination of an on-board, implanted power source in favor of an inductive power scheme can eliminate the need for batteries and the associated battery changes over the patient's life.

In some embodiments, the system 10 can include one or more sensors (not shown), which may be implanted and/or external. For example, the system 10 can include one or more sensors carried by (and implanted with) the neuromodulation device 100. Such sensors can be disposed at any location along the lead 102 and/or electronics package 108. In some embodiments, one, some, or all of the conductive elements 114 can be used for both sensing and stimulation. Use of a single structure or element as the sensor and the stimulating electrode reduces the invasive nature of the surgical procedure associated with implanting the system, while also reducing the number of foreign bodies introduced into a patient. In certain embodiments, at least one of the conductive elements 114 is dedicated to sensing only.

In addition to or instead of inclusion of one or more sensors on the neuromodulation device 100, the system 10 can include one or more sensors separate from the neuromodulation device 100. In some embodiments, one or more of such sensors are wired to the neuromodulation device 100 but implanted at a different location than the neuromodulation device 100. In some embodiments, the system 10 includes one or more sensors that are configured to be wirelessly coupled to the neuromodulation device 100 and/or an external computing device (e.g., control unit 30, user interface 40, etc.). Such sensors can be implanted at the same or different location as the neuromodulation device 100, or may be disposed on the patient's skin.

The one or more sensors can be configured to record and/or detect physiological data (e.g., data originating from the patient's body) over time including changes therein. The physiological data can be used to select certain stimulation parameters and/or adjust one or more stimulation parameters during therapy. Physiological data can include an electromyography (EMG) signal, temperature, movement, body position, electroencephalograph (EEG), air flow, audio data, heart rate, pulse oximetry, eye motion, and/or combinations thereof. In some embodiments, the physiological events can be used to detect and/or anticipate other physiological parameters. For example, the one or more sensors can be configured to sense an EMG signal which can be used to detect and/or anticipate physiological data such as phasic contraction of anterior lingual musculature (such as phasic genioglossus muscle contraction) and measure physiological data such as underlying tonic activity of anterior lingual musculature (such as tonic activity of the genioglossus muscle). Phasic contraction of the genioglossus muscle can be indicative of inspiration, particularly the phasic activity that is layered within the underlying tonic tone of the genioglossus muscle. Changes in physiological data include changes in one or more parameters of a measured signal (e.g., frequency, amplitude, spike rate, etc.), start and end of phasic contraction of anterior lingual musculature (such as phasic genioglossus muscle contraction), changes in underlying tonic activity of anterior lingual musculature (such as changes in tonic activity of the genioglossus muscle), and combinations thereof. In particular, changes in phasic activity of the genioglossus muscle can indicate a respiration or inspiration change and can be used to trigger stimulation. Such physiological data and changes therein can be identified in signals recorded from sensors during different phases of respiration including inspiration. As such, the one or more sensors can include EMG sensors. The one or more sensors can also include, for example, wireless or tethered sensors that measure, body temperature, movement (e.g., an accelerometer), breath sounds (e.g., audio sensors), heart rate, pulse oximetry, eye motion, etc.

In operation, the physiological data provided by the one or more sensors enables closed-loop operation of the neuromodulation device 100. For example, the sensed EMG responses from the genioglossus muscle can enable closed-loop operation of the neuromodulation device 100 while eliminating the need for a chest lead to sense respiration. Operating in closed-loop, the neuromodulation device 100 can maintain stimulation synchronized with respiration, for example, while preserving the ability to detect and account for momentary obstruction. The neuromodulation device 100 can also detect and respond to snoring, for example.

The system 10 can be configured to provide open-loop control and/or closed-loop stimulation to configure parameters for stimulation. In other words, with respect to closed-loop stimulation, the system 10 can be configured to track the patient's respiration (such as each breath of the patient) and stimulation can be applied during or prior to the onset of inspiration, for example. However, with respect to open-loop stimulation, stimulation can be applying without tracking specific physiological data, such as respiration or inspiration. However, even under such an “open loop” scenario, the system 10 can still adjust stimulation and record data, to act on such information. For example, one way the system 10 can act upon such information is that the system 10 can configure parameters for stimulation to apply stimulation in an open loop fashion but can monitor the patient's respiration to know when to revert to applying stimulation on a breath to breath, close-loop fashion such that the system 10 is always working in a closed-looped algorithm to assess data. Treatment parameters of the system may be automatically adjusted in response to the physiological data. The physiological data can be stored over time and examined to change the treatment parameters; for example, the treatment data can be examined in real time to make a real time change to the treatment parameters. In some embodiments, the treatment parameters can be learned from the physiological data stored over time and used to adjust the therapy in real time. This learning can be patient-specific and/or across multiple patients.

Operating in real-time, the neuromodulation device 100 can record data (e.g., via one or more sensors) related to the stimulation session including, for example, stimulation settings, EMG responses, respiration, sleep state including different stages of REM and non-REM sleep, etc. For example, changes in phasic and tonic EMG activity of the genioglossus muscle during inspiration can serve as a trigger for stimulation or changes in stimulation can be made based on changes in phasic and tonic EMG activity of the genioglossus muscle during inspiration or during different sleep states. This recorded data can be uploaded to the user interface 40 and to the remote computing device(s) 80. Also, the patient can be queried to use the interface 40 to log data regarding their perceived quality of sleep, which can also be uploaded to the remote computing device(s) 80. Offline, the remote computing device(s) 80 can execute a software application to evaluate the recorded data to determine whether settings and control parameters can be adjusted to further optimize the stimulation therapy. The software application can, for example, include artificial intelligence (AI) models that learn from recorded therapy sessions how certain adjustments affect the therapeutic outcome for the patient. In this manner, through AI learning, the model can provide patient-specific optimized therapy.

### III. Neuromodulation Devices

FIGS. 2B-2D illustrate various views of the neuromodulation device 100. As previously mentioned, the device 100 can be configured to be implanted at a treatment site within submental and sublingual regions of the patient's head and deliver electrical energy at the treatment site to stimulate the HGN and/or one or more tongue protrusor muscles (e.g., the genioglossus, the geniohyoid, etc.). The device 100 can include an electronics package 108 and a lead 102 coupled to and extending away from the electronics package 108. The lead 102 can comprise a lead body 104 having a plurality of conductive elements 114 and an extension portion 106 extending between the lead body 104 and the electronics package 108. The extension portion 106 can have a proximal end portion 106a coupled to the electronics package 108 via a first connector 110 and a distal end portion 106b coupled to the lead body 104 via a second connector 112. The first connector 110 and/or the second connector 112 can comprise any suitable biocompatible material, such as one or more polymers. For example, the first connector 110 and/or the second connector 112 can include a thermoplastic elastomer, a thermoplastic polyurethane, a silicone, and/or other suitable materials. The material of the first connector 110 and/or the second connector 112 can be a material with high flexibility, good resistance to fluid ingress, low oxidation, good biocompatibility, etc. In some embodiments, the material of the first connector 110 and/or the second connector 112 can be based at least in part on an anatomical environment that the device 100 is configured to be implanted within. For example, an aromatic thermoplastic polyurethane, such as Pellethane™, may be highly hydrophobic and well suited to a wet anatomical environment with substantial interstitial fluid. However, a polycarbonate-based thermoplastic polyurethane, such as Carbothane™, may degrade less than Pellethane™ when positioned within an anatomical environment with substantial amounts of blood, such as in peripheral or subcutaneous environments. Thus, for the device 100 configured to be implanted in sublingual and submental regions, it may be preferable for the first connector 110 and/or the second connector 112 to comprise a polycarbonate-based thermoplastic polyurethane, such as Carbothane™.

The electronics package 108 can be configured to supply electrical current to the conductive elements 114 (e.g., to stimulate) and/or receive electrical energy from the conductive elements 114 (e.g., to sense physiological data). The extension portion 106 of the lead 102 can mechanically and/or electrically couple the electronics package 108 to the lead body 104. The extension portion 106 can comprise a polymeric material such as, but not limited to, a thermoplastic elastomer, a thermoplastic polyurethane, a silicone, or other suitable materials. The extension portion 106 can be sufficiently flexible such that it can bend so as to position the lead body 104 on top of, but spaced apart from, the electronics package 108. As discussed in greater detail below with reference to FIGS. 3A-3F, the neuromodulation device 100 is configured to be implanted within both a submental region and a sublingual region such that the electronics package 108 and lead body 104 are vertically stacked with one or more muscle and/or other tissue layers positioned therebetween. The flexibility of the extension portion 106 enables such a configuration.

In some embodiments, the extension portion 106 comprises a sidewall defining a lumen extending through the extension portion 106. The conductive elements 114 can be electrically coupled to the first antenna 116 and/or the electronics component 118 via one or more electrical connections (also referred to as “electrical conductors” herein) extending through the lumen of the extension portion 106. For example, the proximal end portions of the electrical connections can be routed through the first connector 110 to the electronics component 118 on the electronics package 108. The electrical connections may comprise, for example, one or more wires, cables, traces, vias, and others extending through, on, and/or along the extension portion 106 and lead body 104. The electrical connections can comprise a conductive material such as silver, copper, etc., and each electrical connection can be insulated along all or a portion of its length. In some embodiments, the device 100 includes a separate electrical connection for each conductive element 114. For example, in those embodiments in which the device 100 comprises eight conductive elements 114 (and other embodiments), the device 100 can comprise eight electrical connections, each extending through the lumen of the extension portion 106 from a proximal end at the electronics component 118 to a distal end at one of the conductive elements 114.

In some embodiments, the electronics component 118 comprise an application-specific integrated circuit (ASIC), a discrete electronic component, and/or an electrical connector. In these and other embodiments, the electronics component 118 can comprise, for example, processing and memory components (e.g., microcomputers, microprocessors, computers-on-a-chip, etc.), charge storage and/or delivery components (e.g., batteries, capacitors, electrical conductors) for receiving, accumulating, and/or delivering electrical energy, switching components (e.g., solid state, pulse-width modulation, etc.) for selection and/or control of the conductive elements 114. In some embodiments, the electronics component 118 comprise a data communications unit for communicating with an external device (such as external system 15) via a communication standard such as, but not limited to, near-field communication (NFC), infrared wireless, Bluetooth, ZigBee, Wi-Fi, inductive coupling, capacitive coupling, or any other suitable wireless communication standard. In some examples, the electronics component 118 include one or more processors having one or more computing components configured to control energy delivery via the conductive elements 114 and/or process energy and/or data received by the conductive elements 114 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the conductive elements 114 to obtain data characterizing activity of a patient's muscles. In another example, the functions may involve processing data to determine one or more parameters of the data (e.g., a change in muscle activity, etc.). According to various embodiments, the electronics component 118 can comprise a wireless charging unit for providing power to other electronics component 118 of the device 100 and/or recharging a battery of the device 100 (if included).

The electronics package 108 can also be configured to wirelessly receive energy from a power source to power the neuromodulation device 100. In some embodiments, the electronics package 108 comprises a first antenna 116 configured to wirelessly communicate with the external system 15. As shown in FIG. 2B, in some embodiments the electronics component 118 can be disposed in an opening at a central portion of the first antenna 116. In other embodiments, the electronics component 118 and antenna 116 may have other configurations and arrangements.

The second antenna 12 can be configured to emit an electromagnetic field to induce an electrical current in the first antenna 116, which can then be supplied to the electronics component 118 and/or conductive elements 114. In some embodiments, the first antenna 116 comprises a coil or multiple coils. For example, the first antenna 116 can comprise one or more coils disposed on a flexible substrate. The substrate can comprise a single substrate or multiple substrates secured to one another via adhesive materials. For instance, in some embodiments the substrate comprises multiple layers of a heat resistant polymer (such as polyimide) with adhesive material between adjacent layers. Whether comprising a single layer or multiple layers, the substrate can have one or more vias extending partially or completely through a thickness of the substrate, and one or more electrical connectors can extend through the vias to electrically couple certain components of the electronics package 108, such as the first antenna 116 and/or the previously discussed electronics component 118.

In some embodiments, the first antenna 116 comprises multiple coils. For example, the first antenna 116 can comprise a first coil at a first side of the substrate and a second coil at a second side of the substrate. This configuration can be susceptible to power losses due to substrate losses and parasitic capacitance between the multiple coils and between the individual coil turns. Substrate losses occur due to eddy currents in the substrate due to the non-zero resistance of the substrate material. Parasitic capacitance occurs when these adjacent components are at different voltages, creating an electric field that results in a stored charge. All circuit elements possess this internal capacitance, which can cause their behavior to depart from that of “ideal” circuit elements.

Advantageously, in some embodiments the first antenna 116 can comprise a two-layer, pancake style coil configuration in which the top and bottom coils are configured in parallel. As a result, the coils can generate an equal or substantially equal induced voltage potential when subjected to an electromagnetic field. This can help to equalize the voltage of the coils during use, and has been shown to significantly reduce the parasitic capacitance of the first antenna 116. In this parallel coil configuration, the top and bottom coils are shorted together within each turn. This design has been found to retain the benefit of lower series resistance in a two-coil design while, at the same time, greatly reducing the parasitic capacitance and producing a high maximum power output. Additional details regarding the two-coil configuration can be found in U.S. application Ser. No. 16/866,523, filed May 4, 2020, which is incorporated by reference herein in its entirety.

The first antenna 116 (or one or more portions thereof) can be flexible such that the first antenna 116 is able to conform at least partially to the patient's anatomy once implanted. In some embodiments, the first antenna 116 comprises an outer coating configured to encase and/or support the first antenna 116. The coating can comprise a biocompatible material such as, but not limited to, epoxy, urethane, silicone, or other biocompatible polymers. In some embodiments, the coating comprises multiple layers of distinct materials. In some embodiments, different distinct materials can coat different regions of the first antenna 116. For example, a first material (e.g., epoxy, urethane, silicone, etc.) can coat a first region including the electronics component 118 (e.g., a central region of the first antenna 116) and a second material can coat a second region including the coil turns. As described in greater detail below, in some embodiments the electronics component 118 is within a hermetic enclosure, which can comprise one or more coatings and/or one or more housings, while the coil and/or substrate of the first antenna 116 may or may not be in a hermetic enclosure.

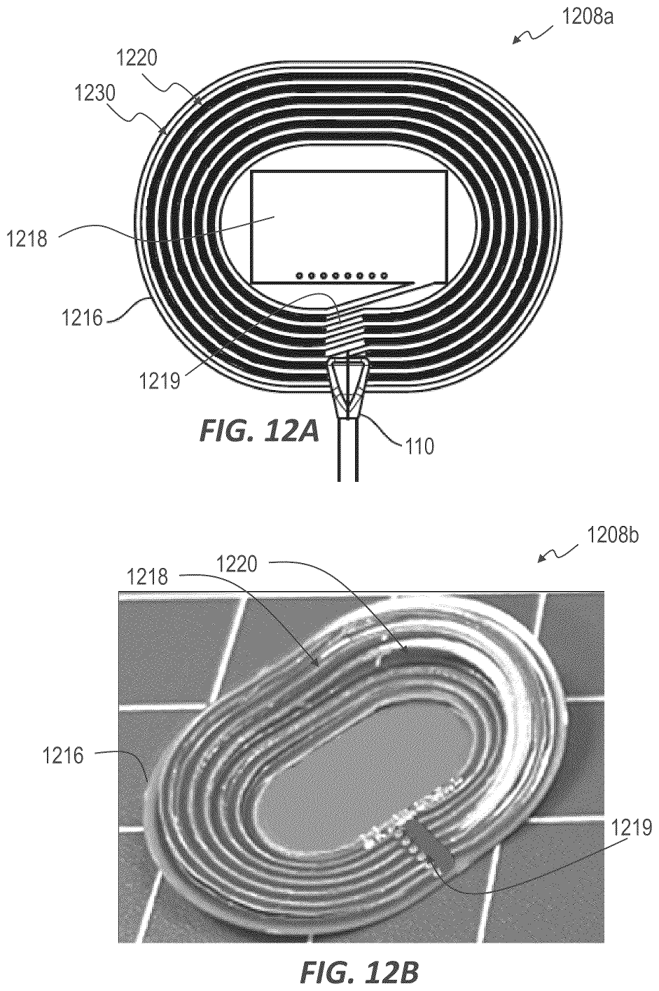

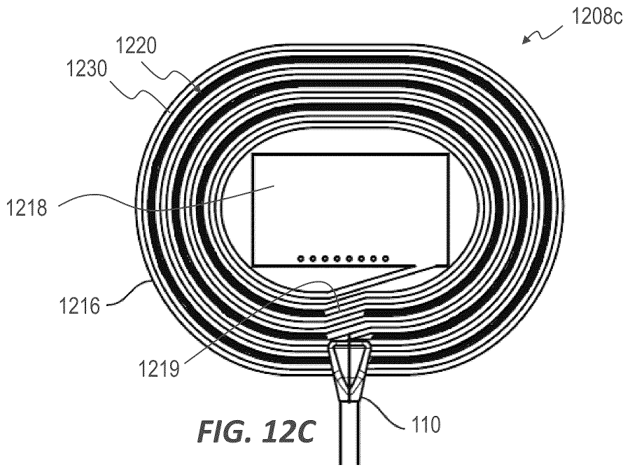

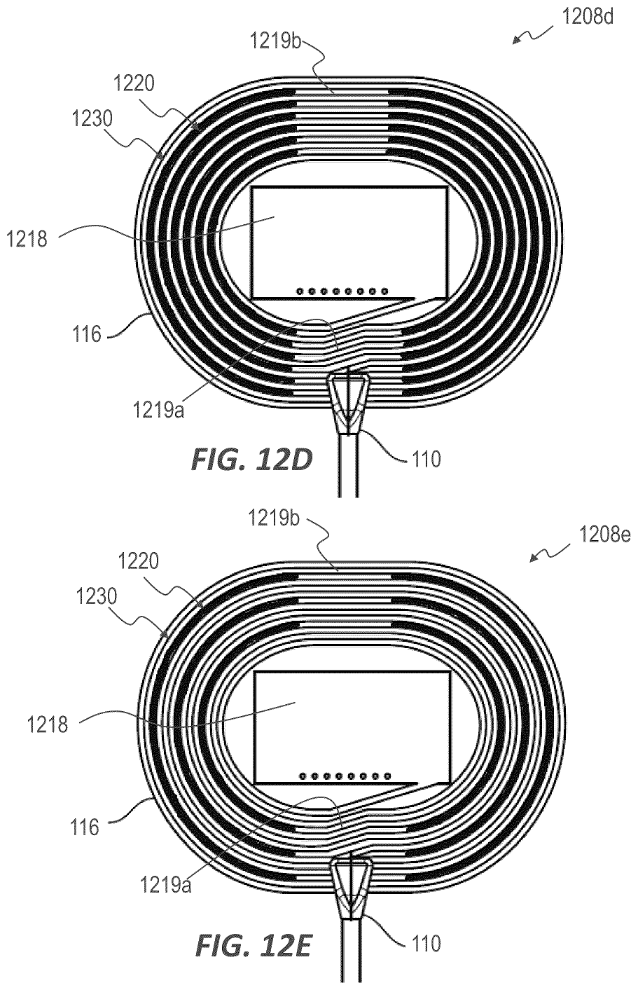

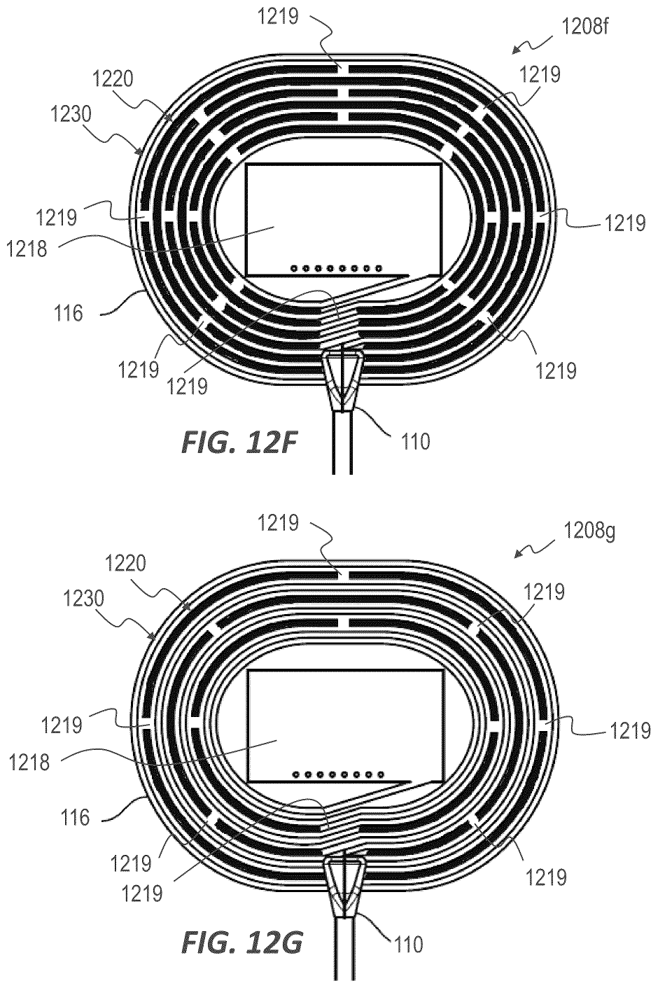

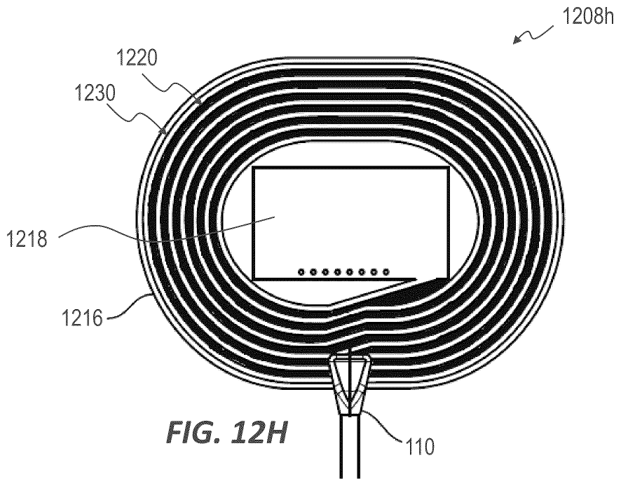

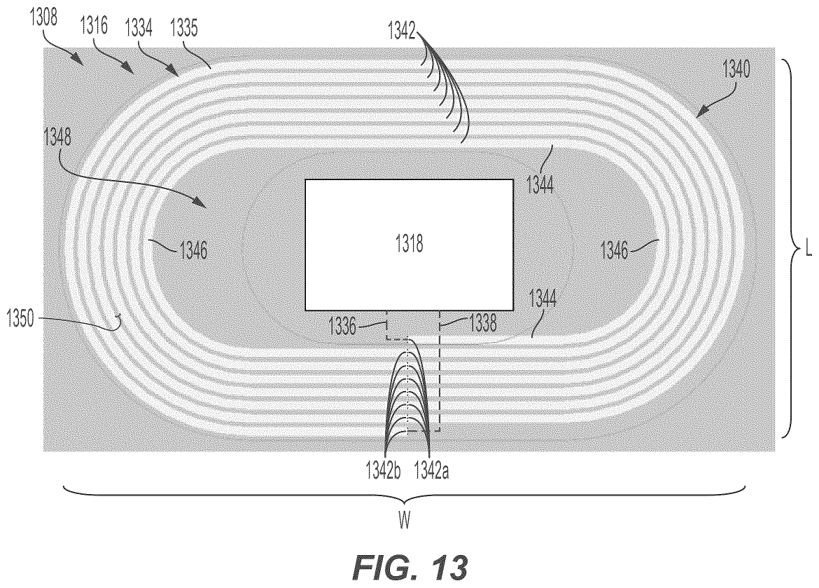

In some embodiments, the first antenna 116 can include one or more open regions (e.g., cuts) through the substrate (or substrates) between coil turns. Such open regions may isolate selected portions of the coil turns and increase movement relative to each other, thereby increasing flexibility and conformability of the overall antenna. The open regions may be formed, for example, by a laser cutting process that removes substrate material in a selected pattern between adjacent coil turns. The first antenna 116 with such open regions can be formed from a single substrate, or can be formed from multiple substrates that are subsequently joined together (e.g., in a suitable overmolding process). As described in further detail below with respect to the examples depicted in FIGS. 12A-12H, in some embodiments the pattern may include one or more open regions where substrate material is removed, thereby partially or fully isolating one or more of the coil turns. Furthermore, in some embodiments the pattern may include one or more strut regions where substrate material remains to help maintain spacing between adjacent coil turns.

For example, FIG. 12A illustrates an example electronics package 1208a with a first antenna 1216 and an electronics component 1218. The first antenna 1216 includes a plurality of coil turns 1230, where a significant circumferential portion of each coil turn 1230 is separated from adjacent coil turns 1230 by an arcuate open region 1220 that extends around the entire coil turn except for a strut region 1219 (e.g., located near the connector 110). For example, the arcuate open regions 1220 can extend continuously around at least 20%, at least 30%, at least 40%, at least 50%, at least 60%, at least 70%, at least 80%, at least 90%, or least 95% of the circumference of an adjacent coil turn. In the example electronics package 1208a, the arcuate open regions 1220 are rotationally aligned such that a single strut region 1219 of substrate material remains. However, in other embodiments some or all of the arcuate open regions may be rotationally offset (e.g., by at least 10 degrees, at least 30 degrees, at least 60 degrees, at least 90 degrees, etc.) such that multiple strut regions 1219 of substrate material remain. FIG. 12B depicts an example electronics package 1208b with a pattern in a first antenna 1216 similar to that shown in FIG. 12A. As shown in FIG. 12B, at least a portion of each coil turn of the antenna 1216 is isolated such that it may move out of the substrate plane relative to adjacent coil turns, at least prior to any substrate coating or covering.

As another example, FIG. 12C illustrates an example electronics package 1208c with a first antenna 1216 and an electronics component 1218. The first antenna 1216 can be similar to the first antenna 1216 of FIG. 12A, except that in the first antenna 1216 of FIG. 12C, every set of two adjacent coil turns 1230 are separated by an adjacent coil turn 1230 by an arcuate open region 1220 that extends around the entire coil turn except for the strut region 1219 (e.g., located near the connector 110). In other words, every other ring (in a radial direction) of substrate material that separates adjacent coil turns can be cut, removed, or otherwise omitted, leaving one or more sets of two adjacent coil turns 1230 circumferentially connected by a ring of substrate material. Accordingly, at least a portion of each circumferentially connected set of coil turn(s) 1230 is isolated such that it may move out of the substrate plane relative to adjacent coil turn(s) 1230, at least prior to any substrate coating or covering. Although FIG. 12C illustrates a first antenna 1216 with arcuate open regions 1220 separating every other ring of substrate material between coil turns 1230, in other embodiments the antenna 1216 may include arcuate open regions 1220 separating any number of circumferentially connected coil turns 1230 (e.g., two connected coil turns, three connected coil turns, etc.). For example, arcuate open regions 1220 may partially isolate sets of three circumferentially connected coil turns 1230, or may partially isolate sets of varying numbers of circumferentially coil turns 1230 (e.g., alternating between partially isolating two connected coil turns and one coil turn).

As another example, FIG. 12D illustrates an example electronics package 1208d with a first antenna 1216 and an electronics component 1218. The first antenna 1216 can be similar to the first antenna 1216 of FIG. 12A, except that in the first antenna 1216 of FIG. 12D, multiple discrete circumferential portions of each coil turn 1230 is separated from adjacent coil turns 1230 by arcuate open regions 1220 that extend around a portion of each coil turn except for strut region 1219a (e.g., located near the connector 110) and strut region 1219b (e.g., located opposite the connector 110 across the antenna 1216). Accordingly, at least two portions of each coil turn 1230 are isolated from adjacent coil turns such that the coil turns 1230 may move out of the substrate plane relative to adjacent coil turn(s) 1230 (e.g., the coil turns 1230 may “butterfly”), at least prior to any substrate coating or covering.

As another example, FIG. 12E illustrates an example electronics package 1208e with a first antenna 1216 and an electronics component 1218. The first antenna 1216 can be similar to the first antenna 1216 of FIG. 12C, except that in the first antenna 1216 of FIG. 12E, multiple discrete circumferential portions of each coil turn 1230 is separated from adjacent coil turns 1230 by arcuate open regions 1220 that extend around a portion of coil turn except for strut region 1219a (e.g., located near the connector 110) and strut region 1219b (e.g., located opposite the connector 110 across the antenna 1216), similar to that described above with respect to FIG. 12D. Accordingly, at least a portion of each circumferentially connected set of coil turn(s) 1230 is isolated such that it may move out of the substrate plane relative to adjacent coil turn(s) 1230, at least prior to any substrate coating or covering. Although FIG. 12E illustrates a first antenna 1216 with arcuate open regions 1220 separating every other ring of substrate material between coil turns 1230, in other embodiments the antenna 1216 may include arcuate open regions 1220 separating any number of circumferentially connected coil turns 1230 (e.g., two connected coil turns, three connected coil turns, etc.). For example, arcuate open regions 1220 may partially isolate sets of three circumferentially connected coil turns 1230, or may partially isolate sets of varying numbers of circumferentially coil turns 1230 (e.g., alternating between partially isolating two connected coil turns and one coil turn).

Furthermore, the cut pattern may define any suitable number of strut regions of substrate material around the coil turns. For example, FIG. 12F illustrates an example electronics package 1208f with a first antenna 1216 similar to the first antenna 1216 of FIG. 12D, except that in the first antenna 1216 of FIG. 12F, multiple discrete circumferential portions of each coil turn 1230 is separated from adjacent coil turns 1230 by arcuate open regions 1220 that extend around a portion of each coil turn except for eight circumferentially-distributed strut regions 1219. As another example, FIG. 12G illustrates an example electronics package 1208g with a first antenna 1216 similar to the first antenna of FIG. 12E, except that in the first antenna 1216 of FIG. 12G, multiple discrete circumferential portions of each set of connected coil turns 1230 are separated from adjacent coil turn(s) 1230 by arcuate open regions 1220 that extend around a portion of each coil turn except for four circumferentially-distributed strut regions 1219. However, in some embodiments the pattern may include one, two, three, four, five, six, seven, eight, nine, ten, or more than ten strut regions arranged equally or unequally around the circumference of the first antenna 1216.

In some embodiments, the first antenna 1216 may include one or more coil turns that are fully circumferentially isolated by an open region (e.g., cut region) (without a strut region 1219). Any of the examples described above with respect to FIGS. 12A-12G can include at least one, two, three, four, five, six, seven, eight, or more than eight coil turns that are fully circumferentially isolated by an open region (e.g., cut region). For example, FIG. 12H illustrates an example electronics package 1208h with a first antenna 1216 and an electronics component 1218. The first antenna 1216 can be similar to the first antenna 1216 of FIG. 12A, except that in the first antenna 1216 of FIG. 12H, every coil turn 1230 is fully circumferentially isolated by an arcuate open region 1220 that extends around the entire coil turn (without a strut region 1219). In other examples, the first antenna 1216 shown in FIGS. 12B-12G can be modified such that any one or more of the coil turns 1230 (or sets of radially adjacent coil turns 1230) are fully circumferentially isolated by arcuate open regions 1220.

In embodiments in which the strut region(s) are present, the pattern of strut regions between adjacent coil turns or adjacent sets of connected coil turn(s) can also include strut regions 1219 that are circumferentially aligned (e.g., as shown in FIG. 12F). Additionally or alternatively, the pattern of strut region(s) 1219 can include strut regions 1219 that are circumferentially offset from one another (e.g., as shown in FIG. 12G), such as by about 15 degrees, about 30 degrees, about 45 degrees (as shown in FIG. 12G), about 60 degrees, about 75 degrees, about 90 degrees, or more than about 90 degrees. Furthermore, the size of strut regions 1219 may vary in any suitable manner depending on, for example, the desired spacing between coil turns 1230. For example, in some embodiments, strut region 1219 may have a width (e.g., arc length around the antenna) of between about 15 μm and about 25 μm, or about 20 μm.

In some embodiments, a region including the electronics component 1218 (e.g., a central region of the first antenna 116) can be coated or otherwise covered by a first material (e.g., epoxy) and a region including the one or more partially or fully isolated coil turns can be coated or otherwise covered by a second material (e.g., urethane, silicone, other polymer of low durometer) configured to enable the coil turns to bend and move. In some embodiments, the region including the coil turns can be overmolded with the second material. For example, any of the example electronics packages described above with respect to FIGS. 12A-12H can include an electronics component 1218 region covered with a first material, and coil turns covered with a second material. In some embodiments, the first material and/or the second material covering at least a portion of the first antenna may help contribute to maintaining spacing between adjacent isolated coil turns (e.g., in embodiments that lack strut regions).

With continued reference to FIGS. 2B-2D, the lead body 104 can comprise a substrate carrying one or more conductive elements 114 configured to deliver and/or receive electrical energy. In some embodiments, the lead body 104 (or one or more portions thereof) comprises flexible tubing with a sidewall defining a lumen. The lead body 104 can comprise a polymeric material such as, but not limited to, a thermoplastic elastomer, a thermoplastic polyurethane, a silicone, or other suitable materials. The lead body 104 can comprise the same material as the extension portion 106 or a different material. The lead body 104 can comprise the same material as the extension portion 106. In some embodiments, the lead body 104 has a different durometer than the extension portion 106. For example, the lead body 104 can have a lower durometer than the extension portion 106, which can enhance patient comfort.

As shown in FIGS. 2B-2D, the lead body 104 has a branched shape comprising a first arm 122 and a second arm 124. To facilitate this configuration, for example, the second connector 112 can be bifurcated and/or branching. The first arm 122 and the second arm 124 can each extend distally and laterally from the second connector 112 and/or the distal end portion 106b of the extension portion 106. The first arm 122 can comprise a proximal portion 122a, a distal portion 122b, and an intermediate portion 122c extending between the proximal portion 112a and the distal portion 122b. Similarly, the second arm 124 can comprise a proximal portion 124a, a distal portion 124b, and an intermediate portion 124c extending between the proximal portion 124a and the distal portion 124b. In some embodiments, the first arm 122 can comprise a cantilevered, free distal end 123 and/or the second arm 124 can comprise a cantilevered, free distal end 125. The first arm 122 and/or the second arm 124 can include one or more fixation elements 130, for example the fixation elements 130 shown at the distal portions 122b, 124b of the first and second arms 122, 124 in FIGS. 2B-2D. The fixation elements 130 can be configured to securely, and optionally releasably, engage patient tissue to prevent or limit movement of the lead body 104 relative to the tissue.

While being flexible, the lead 102 and/or one or more portions thereof (e.g., the lead body 104, the extension portion 106, etc.) can also be configured to maintain a desired shape. This feature can, for example, be facilitated by electrical conductors that electrically connect the conductive elements 114 carried by the lead body 104 to the electronics package 108, by an additional internal shape-maintaining (e.g., a metal, a shape memory alloy, etc.) support structure (not shown), by shape setting the substrate comprising the lead 102, etc. In any case, one or more portions of the lead 102 can have a physical property (e.g., ductility, elasticity, etc.) that enable the lead 102 to be manipulated into a desired shape or maintain a preset shape. Additionally or alternatively, the lead 102 and/or one or more portions thereof (e.g., the lead body 104, the extension portion 106, etc.) can be sufficiently flexible to at least partially conform to a patient's anatomy once implanted and/or to enhance patient comfort.

The conductive elements 114 can be carried by the sidewall of the lead body 104. For example, the conductive elements 114 can be positioned on an outer surface of the sidewall and/or within a recessed portion of the sidewall. In some embodiments, one or more of the conductive elements 114 is positioned on an outer surface of the sidewall and extends at least partially around a circumference of the sidewall. The lumen of the lead body 104 can carry one or more electrical conductors that extend through the lumen of the lead body 104 and the lumen of the extension portion 106 from the conductive elements 114 to the electronics package 108. The sidewall can define one or more apertures through which an electrical connector can extend.

As previously described, the conductive elements 114 can be connected to electronics package 108 via one or more electrical conductors. The electrical conductors can be positioned on the sidewall of the lead 102 (e.g., the extension portion 106 and/or the lead body 104) and/or within a lumen of the lead 102. In some embodiments in which the electrical conductors are positioned within the lumen of the extension portion 106 of the lead 102, the lumen can be backfilled once the electrical conductors have been positioned within the lumen. The lumen can be backfilled with an adhesive and/or an elastomer. In some embodiments, the lumen is backfilled with a silicone adhesive, for example. In some embodiments, the extension portion 106 can be injection molded around the electrical conductors. Backfilling the lumen and/or injection molding the extension portion 106 around the electrical conductors can fill space within the lumen of the extension portion 106 otherwise not occupied by the electrical conductors, which may, for example, help prevent or limit fluid from entering the lead 102 and corroding or degrading the electrical conductors.

In some embodiments, each conductive element 114 is connected to one respective electrical conductor such that the number of electrical conductors equals the number of conductive elements 114. Still, in some embodiments, the device can include more or fewer electrical conductors than conductive elements 114 (e.g., an electrical conductor can be connected to multiple conductive elements 114). A conductive element 114 can be connected to an electrical conductor via welding, soldering, and/or any other suitable technique for forming an electrical and/or mechanical connection between the conductive element 114 and the electrical conductor. For example, the conductive element 114 can be connected to an electrical conductor via tack welding. The conductive element 114 can be connected to the respective electrical conductor at one or more locations along a length of the electrical conductor.

In some embodiments, a material and/or configuration of an electrical conductor can be selected based on a desired mechanical performance of the electrical conductor. For example, a stranded electrical conductor may have better flexibility and fatigue resistance than a solid core wire, which may be desirable for use in the human body. In some embodiments, it may be advantageous for the electrical conductors to comprise a material having a low resistivity, as such electrical conductors may draw less power than equivalent electrical conductors with higher resistivity. An electrical conductor of the present technology can comprise any suitable metal such as titanium, chromium, niobium, tantalum, vanadium, zirconium, aluminum, cobalt, nickel, stainless steels, or alloys of any of the foregoing metals.

Each of the conductive elements 114 may comprise an electrode, an exposed portion of a conductive material, a printed conductive material, and other suitable forms. In some embodiments, one or more of the conductive elements 114 comprises a ring electrode. The conductive elements 114 can be crimped, welded, adhered to, or positioned over an outer surface and/or recessed portion of the lead body 104. Additionally or alternatively, each of the conductive elements 114 can be welded, soldered, crimped, or otherwise electrically coupled to a corresponding electrical conductor. In some embodiments, one or more of the conductive elements 114 comprises a flexible conductive material disposed on the lead body 104 via printing, thin film deposition, or other suitable techniques. Each one of the conductive elements 114 can comprise any suitable conductive material including, but not limited to, platinum, iridium, silver, gold, nickel, titanium, copper, combinations thereof, and/or others. For example, one or more of the conductive elements 114 can be a ring electrode comprising a platinum iridium alloy. In some embodiments, one or more of the conductive elements 114 comprises a coating configured to improve biocompatibility, conductivity, corrosion resistance, surface roughness, durability, or other parameter(s) of the conductive element 114. As but one example, one or more of the conductive elements 114 can comprise a coating of titanium and nitride.

In some embodiments, one or more conductive elements 114 has a length of about 1 mm. Additionally or alternatively, one or more conductive elements 114 can have a length of about 0.25 mm, about 0.5 mm, about 0.75 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. In any case, adjacent conductive elements 114 carried by one of the first or second arms 122, 124 can be spaced apart along a length of the arm by about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. The conductive elements 114 can have the same length or different lengths.

Furthermore, while the device 100 shown in FIGS. 2B-2D includes conductive elements 114 that are generally equally spaced apart from each other on the first arm 122 and on the second arm 124, other distributions of conductive elements 114 are within the scope of the present technology. For example, on the first arm 122 and/or the second arm 124, at least a portion of the conductive elements 114 can be equally spaced apart along the length of the arm, and/or at least a portion of the conductive elements 114 can be unequally spaced apart along the length of the arm.

For example, in some embodiments with unequal spacing of conductive elements 114, the spacing between conductive elements 114 along the first arm 122 and/or the second arm 124 can decrease in a proximal-to-distal direction (e.g., conductive elements 114 located at a distal portion of a lead body arm 122, 124 can be located closer to each other compared to conductive elements 114 located at a proximal portion of the lead body arm). As another example, in some embodiments with unequal spacing of conductive elements 114, the spacing between conductive elements 114 along the first arm 122 and/or the second arm 124 can increase in a proximal-to-distal direction (e.g., conductive elements 114 located at a distal portion of a lead body arm 122, 124 can be located farther from each other compared to conductive elements 114 located at a proximal portion of the lead body arm). As another example, in some embodiments with unequal spacing of conductive elements 114, the spacing between conductive elements 114 along the first arm 122 and/or the second arm 124 can regularly alternate between a first distance and a second distance, where the first and second distances are different. As another example, in some embodiments with unequal spacing of conductive elements 114, the spacing between conductive elements 114 along the first arm 122 and/or the second arm 124 can be irregular or random.

The spacing or distribution of conductive elements 114 on the first arm 122 can mirror that of conductive elements 114 on the second arm 124, or the spacing or distribution of conductive elements 114 can be different on the first arm 122 compared to the second arm 124.

While the device 100 shown in FIGS. 2B-2D includes eight conductive elements 114 (four conductive elements 114 carried by the first arm 122 and four conductive elements 114 carried by the second arm 124), other numbers and configurations of conductive elements 114 are within the scope of the present technology. For example, the first arm 122 can carry the same number of conductive elements 114 as the second arm 124, or the first arm 122 can carry a different number of conductive elements 114 as the second arm 124 (e.g., the first arm 122 can carry more or fewer conductive elements 114 than the second arm 124). The first arm 122 and/or the second arm 124 can carry one conductive element 114, two conductive elements 114, three conductive elements 114, four conductive elements 114, five conductive elements 114, six conductive elements 114, seven conductive elements 114, eight conductive elements 114, nine conductive elements 114, ten conductive elements 114, or more than ten conductive elements 114. In some embodiments, one of the first arm 122 or the second arm 124 does not carry any conductive elements 114.

The conductive elements 114 can be configured for stimulation and/or sensing. Stimulating conductive elements 114 can be configured to deliver energy to an anatomical structure, such as, for example, a nerve or muscle. In some embodiments, the conductive elements 114 are configured to deliver energy to a hypoglossal nerve of a patient to increase the activity of the patient's tongue protrusor muscles. Sensing conductive elements 114 can be used obtain data characterizing a physiological activity of a patient (e.g., muscle activity, temperature, etc.). In some embodiments, the sensing conductive elements 114 are configured to detect electrical energy produced by a muscle of a patient to obtain EMG data characterizing an activity of the muscle. In some embodiments, the sensing conductive elements are configured to measure impedance across the conductive elements. As but one example, in some embodiments the conductive elements 114 are configured to deliver energy to a hypoglossal nerve of a patient to increase activity of the genioglossus and/or geniohyoid muscles, and obtain EMG data characterizing activity of the genioglossus muscle and/or the geniohyoid muscle of the patient. Still, the conductive elements 114 can be configured to deliver energy to and/or measure physiological electrical signals from other patient tissues.

The function that each of the conductive elements 114 is configured to perform (e.g., delivering energy to patient tissue, receiving energy from patient tissue, etc.) can be controlled by a processor of the electronics component 118 of the electronics package 108. In some embodiments, one or more of the conductive elements 114 is configured for only one of delivering energy to patient tissue or receiving energy from patient tissue. In various embodiments, one or more of the conductive elements 114 is configured for both delivering energy to patient tissue and receiving energy from patient tissue. In some embodiments, the functionality of a conductive element 114 can be based, at least in part, on an intended positioning of the device 100 within a patient and/or the position of the conductive element 114 on the lead body 104. One, some, or all of the conductive elements 114 can be positioned relative to patient tissue, such as nerves and/or muscles, so that it may be desirable for the conductive element(s) 114 to be able to both deliver energy to the patient tissue and receive energy from the patient tissue. Additionally or alternatively, some conductive elements 114 can have an intended position relative to specific patient tissues so that only delivery of stimulation energy is desired while other conductive elements 114 can have an intended position relative to specific patient tissues so that only receipt of sensing energy is desired. Advantageously, the configurations of the conductive elements 114 can be configured in software settings (which can be facilitated by electronics component 118 of the electronics package 108) so that the configurations of the conductive elements 114 are easily modifiable.

Whether configured for stimulating and/or sensing, each of the conductive elements 114 can be configured and used independently of the other conductive elements 114. Because of this, all or some of conductive elements 114, whichever is determined to be most effective for a particular implementation, can be utilized during the application of stimulation therapy. For example, one conductive element 114 of the first arm 122 can be used as a cathode while one conductive element 114 of the second arm 124 is used as an anode (or vice versa), two or more conductive elements 114 of the first arm 122 can be used (one as the cathode and one as the anode) without use of any conductive elements 114 of the second arm 124 (or vice versa), multiple pairs of conductive elements 114 of the first and second arms 122, 124 can be used, or any other suitable combination. As discussed in greater detail below, the conductive element(s) 114 used for sensing and/or stimulation can be selected based on desired data to be collected and/or desired modulation of neural or muscle activity. For example, specific pairs of the conductive elements 114 can be used for creating an electric field tailored to stimulation of certain regions of the muscle and/or HGN that causes favorable changes in tongue position and/or pharyngeal dilation. Additionally or alternatively, conductive element(s) 114 that are positioned in contact with muscle tissue when the device 100 is implanted may be more favorable to use for EMG sensing than conductive element(s) 114 that are not positioned in contact with muscle tissue.

The lead body 104 can have a shape configured to facilitate delivery of electrical energy to a specific treatment location within a patient and/or detection of electrical energy from a sensing location within the patient. The conductive elements 114 carried by the first arm 122 can be configured to deliver electrical stimulation energy to one hypoglossal nerve (e.g., the right or the left hypoglossal nerve) of a patient and the conductive elements 114 carried by the second arm 124 can be configured to deliver electrical stimulation energy to the other hypoglossal nerve (e.g., the other of the right or the left hypoglossal nerve) of the patient.

Without being bound by theory, it is believed that increased activity of the tongue protrusor muscles during sleep reduces upper airway resistance and improves respiration. Thus, devices of the present technology are configured to deliver stimulation energy to motor nerves that control the tongue protrusors. In some embodiments, the device 100 is configured to deliver stimulation energy to the hypoglossal nerve to cause protrusion of the tongue. Additionally or alternatively, the device 100 can be configured to receive sensing energy produced by activity of one or more muscles of a patient (such as the genioglossus muscle), which can be used for closed-loop delivery of stimulation energy, evaluation of patient respiration, etc.

The device can be configured to be implanted at an anatomical region of a patient that is bound anteriorly and laterally by the patient's mandible, superiorly by the superior surface of the tongue, and inferiorly by the patient's platysma. Such an anatomical region can include, for example, a submental region and a sublingual region. The sublingual region is bound superiorly by the oral floor mucosa and inferiorly by the mylohyoid and includes the plane between the genioglossus muscle and the geniohyoid muscle. The submental region is bound superiorly by the mylohyoid and inferiorly by the platysma muscle. FIGS. 3A-3F depict various views of the device 100 implanted within a patient. As shown in FIGS. 3A-3F, the neuromodulation device 100 is configured to be positioned such that the electronics package 108 is disposed on or near the inferior surface of the mylohyoid in a submental region while the lead body 104 is positioned between the geniohyoid and genioglossus in a sublingual region with the arms 122, 124 disposed along the left and right hypoglossal nerves. The arms 122, 124 can be positioned such that the conductive elements 114 are disposed near the portions of the distal arborization of the hypoglossal nerves that innervate the genioglossus. In particular, the conductive elements 114 can be positioned proximate the portions of the distal arborization that innervate the horizontal fibers of the genioglossus while limiting and/or avoiding stimulation of the portions of the distal arborization of the hypoglossal nerve that activate retrusor muscles. When implanted, the extension portion 106 of the lead 102 can extend in an anterior direction away from the electronics package 108 (towards the mandible), then bend superiorly and extend through the geniohyoid muscle until bending back posteriorly and extending within a tissue plane between the geniohyoid and genioglossus muscles. In some embodiments, the extension portion 106 straddles the right and left geniohyoid muscles.

In some embodiments, at least a portion of the electronics package 108 can be implanted between one or more muscles and/or between layers of one or more muscles. For example, at least a portion of the electronics package 108 can be configured to be placed proximate to (e.g., adjacent to) the mylohyoid and/or one or more digastric muscles. The electronics package 108 can be sufficiently flexible so that, once implanted, the electronics package 108 at least partially conforms to the curvature of the mylohyoid. Additionally or alternatively, the electronics package 108 can have a shape reflecting the curvature of the mylohyoid. In some embodiments, the electronics package 108 can comprise fixation elements (similar to fixation elements 130, securing elements 1132, or otherwise) that are configured to engage the mylohyoid (and/or other surrounding tissue) and prevent or limit motion of the electronics package 108 once implanted.

Additionally or alternatively, the electronics package 108 can be configured to be held by a digastric muscle (e.g., an anterior belly of a digastric muscle, a posterior belly of a digastric muscle, etc.) when the electronics package 108 is placed in a patient. The digastric muscle can, for example, help improve the implantation and/or fixation of the electronics package 108 adjacent to and/or inferior to the mylohyoid. In some variations, the implanted electronics package 108 can be placed in a manner adjacent to a digastric muscle such that the electronics package 108 is configured to receive pressure by the digastric muscle, due at least in part to the physical presence and natural tone of the digastric muscle. Additionally or alternatively, in some variations, the electronics package 108 can be held in place by one or more fixation elements configured to interact with the digastric muscle and/or one or more surrounding anatomical structures. For example, in some embodiments, the electronics package 108 can include or be coupled to one or more mechanical fasteners (e.g., a clip, clamp, staple, tine, hook, barb, anchor, suture, fixation elements 130, securing elements 1132, and/or other suitable fasteners) that are configured to engage the digastric muscle and/or surrounding tissue to prevent or limit motion of the electronics package 108. In some embodiments, one or more fixation points (e.g., a screw, a plate) and/or surgical anchors (e.g., a bone anchor) utilizing one or more surrounding tissues are used to hold the electronics package 108. In some embodiments, the one or more fasteners configured to engage one or more soft tissues surrounding the electronics package 108 can include a flexible loop that surrounds at least a portion of the one or more soft tissues (e.g., a digastric muscle tendon). Fixation of the electronics package 108 by the digastric muscle and/or surrounding tissues can be temporary (e.g., during an implantation procedure) or long-term (e.g., after implantation). The electronics package 108 can be configured to be held unilaterally or bilaterally by the digastric muscle.

The lead body 104 can be configured to be positioned between the genioglossus and geniohyoid muscles of a patient so that the conductive elements 114 are positioned proximate the hypoglossal nerve. Although not shown in FIGS. 3A-3F, the hypoglossal nerve is located between the genioglossus and fascia and/or fat located between the genioglossus and the geniohyoid. In some embodiments, the lead body 104 is configured to be positioned at or just inferior to the fat between the hypoglossal nerve and the geniohyoid and thus is not positioned in direct contact with the hypoglossal nerve. In any case, once the device 100 is implanted, the lead body 104 can extend posteriorly away from the distal end portion 106b of the extension portion 106. The lead body 104 can then branch or diverge laterally such that the first arm 122 of the lead body 104 is positioned proximate one of the patient's hypoglossal nerves and the second arm 124 is positioned proximate the contralateral hypoglossal nerve. The fixation elements 130 can engage patient tissue (e.g., the fat underlying the hypoglossal nerves, etc.) to prevent or limit motion of the first and second arms 122, 124 relative to the patient tissue.

As best shown in FIG. 3C, and as described in greater detail below, the arms 122, 124 of the lead body 104 can bend out of the plane of the extension portion 106, in addition to extending laterally away from the extension portion 106, such that the arms 122, 124 outline a somewhat concave shape. Advantageously, this concave shape can accommodate the convex inferior surface of the genioglossus and still keep the arms 122, 124 positioned near the distal arborization of the hypoglossal nerve.

In some embodiments, conductive elements 114 are selected for use that selectively activate the protrusor muscles of a patient. In these and other embodiments, the specific positioning of the first and second arms 122, 124 relative to specific branches of the hypoglossal nerves need not be identified prior to stimulation of desired portions of the nerve and/or muscles. For example, in embodiments in which the lead body 104 includes more than two conductive elements 114, the combination of conductive elements 114 that is used for treating a patient can be selected based on physiological responses to test stimulations. For example, stimulation energy can be delivered to the hypoglossal nerve(s) via multiple combinations of conductive elements 114 and a physiological response (e.g., EMG data, tongue position, pharyngeal opening size, etc.) and/or a functional outcome (e.g., Fatigue Severity Scale, Epworth Sleepiness Scale, etc.) can be evaluated for each combination. Based on the evaluation(s), the conductive elements 114 that are selected to deliver stimulation energy can be conductive elements 114 that are associated with favorable responses/outcomes.

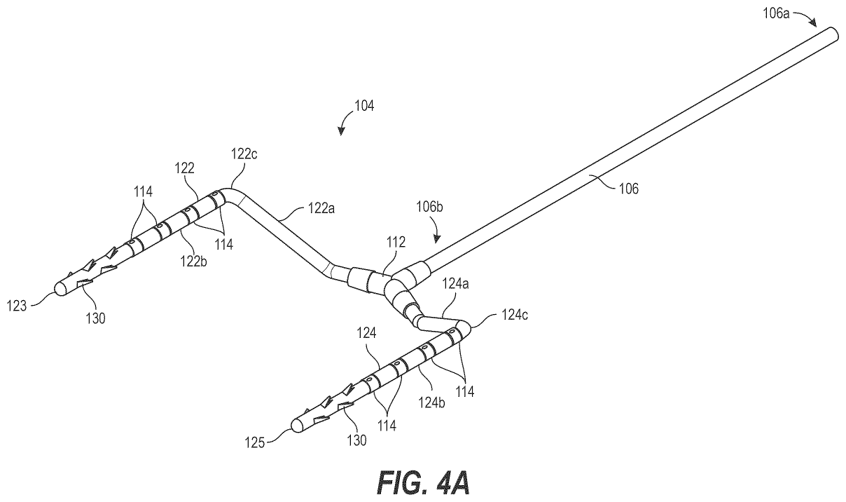

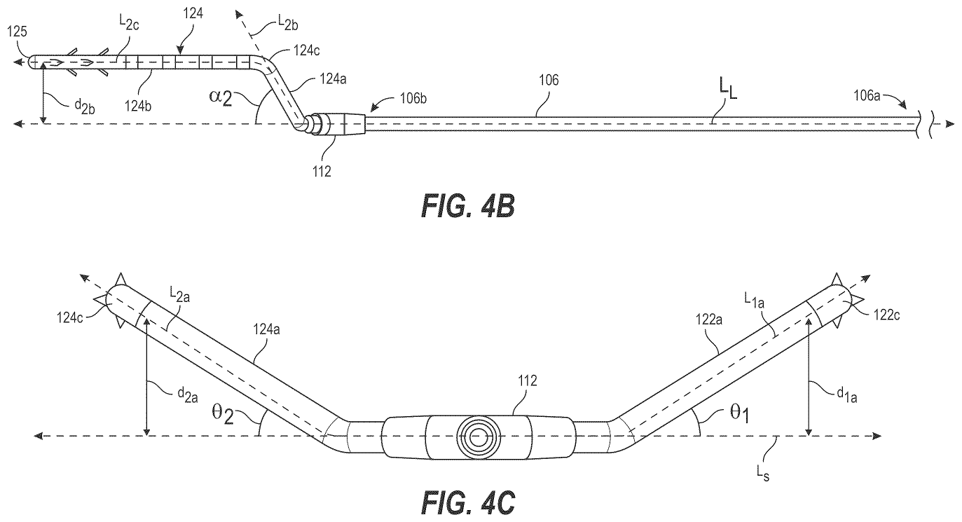

The shape of the lead body 104 can facilitate electrical coupling between the conductive elements 114 and the hypoglossal nerves of a patient. FIGS. 4A-4C are perspective, side, and end views, respectively, of the lead 102 isolated from the electronics package 108 and first connector 110 for further discussion of the lead body 104 shape. With reference to FIGS. 3A-4C, the first and second arms 122, 124 can branch distally and laterally away from the distal end portion 106b of the extension portion 106. As shown in FIGS. 4B and 4C, the proximal portion 122a of the first arm 122 can extend laterally away from the distal end portion 106b of the extension portion 106 in a first lateral dimension L₁ₐ and the proximal portion 124a of the second arm 124 can extend laterally away from the distal end portion 106b of the extension portion 106 in a second lateral dimension L₂ₐ. Extension of the proximal portions 122a, 124a in diverging lateral dimensions L₁ₐ, L₂ₐ enables positioning of the first and second arms 122, 124 bilaterally within the patient such that each of the first and second arms 122, 124 is positioned proximate one of the right hypoglossal nerve or the left hypoglossal nerve. As shown in FIG. 4B, the proximal portion 124a of the second arm 124 can extend distally away from the distal end portion 106b of the extension portion 106 and/or the second connector 112 in a horizontal dimension L_{2b} angled with respect to a longitudinal axis L_{L} of the lead 102 according to an angle α2. The longitudinal axis L_{L} of the lead 102 can be aligned with the extension portion 106 of the lead 102 (e.g., as shown in FIG. 4B) or may be offset from the extension portion 106.

As shown in FIG. 4C, the proximal portion 122a of the first arm 122 can be angled away from a lateral axis L_{S} of the lead 102 by a first angle θ1 such that the proximal portion 122a is spaced apart from the lateral axis L_{S} by a first distance dia. The first distance d₁ₐ can increase proximally to distally and/or can increase with increasing lateral distance from the distal end portion 106b of the extension portion 106 and/or the second connector 112. As shown in FIGS. 4B and 4C, the proximal portion 124a of the second arm 124 can be angled away from the lateral axis L_{S} of the lead 102 by a second angle θ2 (which can be the same or different than the first angle θ1) such that the proximal portion 124a is spaced apart from the lateral axis L_{S} by a second distance d₂ₐ. The second distance d₂ₐ can increase proximally to distally and/or can increase with increasing lateral distance from distal end portion 106b of the extension portion 106 and/or the second connector 112.

The distal portion 122b of the first arm 122 can extend distally away from the intermediate portion 122c in a first longitudinal dimension (not shown) and the distal portion 124b of the second arm 124 can extend distally away from the intermediate portion 124c of the second arm 124 in a second longitudinal dimension L_{2c}. In some embodiments, the first longitudinal dimension and/or the second longitudinal dimension L_{2c} can be substantially parallel to the longitudinal axis L_{L} of the lead 102. In any case, the distal portion 124b of the second arm 124 can be spaced apart from the longitudinal axis of the lead L_{L} by a vertical distance d_{2b}. Similarly, the distal portion 122b of the first arm 122 can be spaced apart from the longitudinal axis of the lead L_{L} by a vertical distance.

The distal portion 122b of the first arm 122 and/or the distal portion 124b of the second arm 124 can be positioned in a different plane and/or at a different elevation than the extension portion 106. Angling the proximal portions 122a, 124a of the arms 122, 124 vertically away from the extension portion 106 facilitates establishing sufficient and stable electrical coupling of the conductive elements 114 with the fat underlying the hypoglossal nerves. As shown in FIGS. 3B-3F, the distal end portion 106b of the extension portion 106 of the lead can be configured to be positioned at, near, and/or just superior to the geniohyoid when implanted. However, because of the branched and angled structure of the lead body 104, the lead body 104 can extend superiorly towards the genioglossus. Specifically, the proximal portions 122a, 124a of the arms 122, 124 can extend superiorly. In some embodiments, when the device 100 is implanted, the genioglossus (and the underlying hypoglossal nerve branches, fascia, fat, etc.) can rest on the first and second arms 122, 124 of the lead body 104, which can facilitate electrical contact with between the conductive elements 114 and the patient tissue.

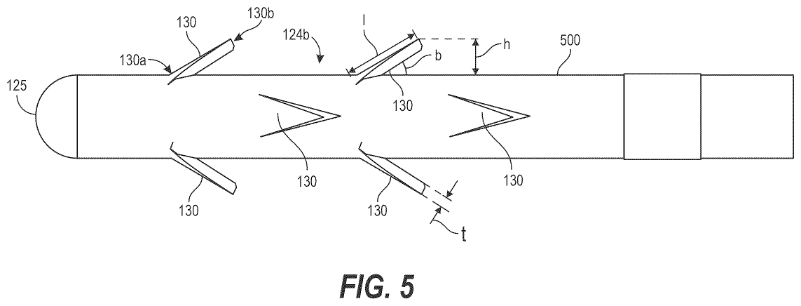

The device 100 can include fixation elements 130 configured to engage patient tissue to secure the device 100 to the tissue. For example, fixation elements 130 of the lead body 104 can further facilitate engagement of the lead body 104 with patient tissue. FIG. 5 is an enlarged side view of the distal portion 124b of the second arm 124 and corresponding example fixation elements 130. One or more of the fixation elements 130 can extend from a first end portion 130a at the outer surface of a sidewall 500 of the lead to a second end portion 130b that is radially spaced apart from the outer surface of the sidewall. In other words, the second end portion 130b can be radially spaced apart from a cylindrical outer surface of the sidewall 500. Each fixation element 130 can have a length l defined between the first and second end portions 130a, 130b of the fixation element 130 and a thickness t. In some embodiments, the length l of one or more of the fixation elements 130 is between about 0.7 mm to about 1.5 mm, between about 0.8 mm and about 1.4 mm, between about 0.9 mm and about 1.3 mm, between about 1.0 mm and about 1.2 mm, about 0.7 mm, about 0.8 mm, about 0.9 mm, about 1.0 mm, about 1.1 mm, about 1.2 mm, about 1.3 mm, about 1.4 mm, or about 1.5 mm. In some embodiments, the thickness t of one or more of the fixation elements 130 is between about 0.1 mm and about 0.5 mm, between about 0.2 mm and about 0.4 mm, about 0.1 mm, about 0.2 mm, about 0.3 mm, about 0.4 mm, or about 0.5 mm. In some embodiments, the thickness t can be based on and/or substantially equal to a thickness of the sidewall 500 of the lead body. In some embodiments, the thickness t may vary (e.g., taper in thickness from the first end portion 130a to the second portion 130b). The second end portion 130b can be spaced apart from the sidewall 500 by a height h such that the fixation element 130 is angled with respect to the sidewall by an angle b. The height h can be no more than 1 mm, no more than 0.75 mm, no more than 0.5 mm, no more than 0.4 mm, no more than 0.3 mm, no more than 0.2 mm, no more than 0.1 mm, about 1 mm, about 0.5 mm, about 0.1 mm, or more than 1 mm. According to various embodiments, the angle b can be less than 90 degrees, for example about 80 degrees, about 75 degrees, about 70 degrees, about 65 degrees, about 60 degrees, about 55 degrees, about 50 degrees, about 45 degrees, about 40 degrees, about 35 degrees, about 30 degrees, about 25 degrees about 20 degrees, about 15 degrees, or about 10 degrees. Although the fixation elements 130 are shown in FIG. 5 as having a generally linear profile along their length 1, in other embodiments one or more fixation elements 130 can have any suitable profile, such as curved (e.g., concave, convex, etc.). The fixation elements 130 can be configured to engage patient tissue (e.g., the fat underlying the hypoglossal nerve, muscle tissue, etc.) to prevent or limit motion of one or more portions of the device 100 relative to the tissue. Any of the fixation elements 130 disclosed herein can be configured to prevent or limit movement of the portion of the device in an anterior direction, a posterior direction, a medial direction, a lateral direction, a superior direction, and/or an inferior direction.

Any portion of the device 100 can comprise fixation elements 130. For example, the proximal portion 122a of the first arm 122, the distal portion 122b of the first arm 122, the intermediate portion 122c of the first arm 122, the proximal portion 124a of the second arm 124, the distal portion 124b of the second arm 124, the intermediate portion 124c of the second arm 124, the extension portion 106, the electronics package 108, and/or another suitable portion of the device 100 can comprise fixation elements 130. In some embodiments, the device 100 comprises fixation elements 130 positioned between adjacent conductive elements 114. For example, one or more fixation elements 130 can be positioned between a distalmost conductive element 114 of an arm and an adjacent conductive element 114 of the arm, between a proximalmost conductive element 114 of an arm and an adjacent conductive element 114 of the arm, between intermediate conductive elements 114 between the distalmost and proximalmost conductive elements 114, etc. In some embodiments, for example as shown in FIG. 5, the fixation elements 130 can be disposed at the distal end portion of one or both of the arms 122, 124, for example between the distalmost conductive element 114 and the distal tip of the respective arm. Because a weight and/or a stiffness of the device 100 may be greater at the electronics package 108 and/or one or more regions of the extension portion 106 than at the distal end portion of the arms 122, 124, the arms 122, 124 may tend to displace away from the fat pads near the hypoglossal nerve during implantation of the device 100. However, such distal positioning of the fixation elements 130 can allow the arms 122, 124 to better grab the fat pads and remain at their intended locations during implantation of the device 100. In some embodiments, one or more fixation elements 130 can be positioned proximal of a proximalmost conductive element 114 of a given arm, for example at or near the intermediate portion of the arm and/or the proximal portion of the arm.

Although FIG. 5 depicts six fixation elements 130 carried by the distal portion 124b of the second arm 124, other numbers of fixation elements 130 are possible. For example, the distal end portion of each arm can include one fixation element 130, two fixation elements 130, three fixation elements 130, four fixation elements 130, five fixation elements 130, six fixation elements 130, seven fixation elements 130, eight fixation elements 130, nine fixation elements 130, ten fixation elements 130, eleven fixation elements 130, twelve fixation elements 130, and/or more than twelve fixation elements 130. However, it may be desirable in some applications to limit the number of fixation elements 130 carried by each arm. For example, it may be desirable to use fewer fixation elements 130 so that the arm can releasably engage the tissue. If an arm includes too many fixation elements 130, the arm may not be able to separate from the tissue after the fixation elements 130 have engaged the tissue without causing trauma to the tissue. In some embodiments, it may be desirable to reposition the arm after the fixation elements 130 have engaged the tissue, for example to move the conductive elements 114 to a more favorable position relative to the HGN. Limiting the number of fixation elements 130 per arm can provide the desired balance between secure engagement of the arm with the tissue while still allowing the arm to be separated from the tissue after the fixation elements 130 have engaged the tissue. In some embodiments, each arm can comprise no more than eight fixation elements 130, for example, two fixation elements 130, four fixation elements 130, six fixation elements 130, or eight fixation elements 130.

Additionally or alternatively, it may desirable to limit the lengths of the distal portions 122b, 124b of the arms 122, 124, which can constrain the number of fixation elements 130 that the distal portions 122b, 124b of the arms 122, 124 include. For example, it may be desirable for a distance between the distalmost conductive element 114 and the distal tip of a respective arm to be less than about 12 mm, less than about 11 mm, less than about 10 mm, less than about 9 mm, less than about 8 mm, less than about 7 mm, or less than about 6 mm to prevent or limit the distal tip of the arm from inadvertently contacting the hyoid bone or other anatomical structures (e.g., bones, muscles, nerves, etc.) when the conductive elements 114 are aligned with the HGN.

Some or all of the fixation elements 130 can be distributed around a circumference of the arm or can be aligned circumferentially. Additionally or alternatively, some or all of the fixation elements 130 can be spaced apart along a length of the arm or can be aligned axially along the length of the arm. For example, in some embodiments the fixation elements 130 comprise a first set of fixation elements and a second set of fixation elements. The first set of fixation elements can be circumferentially arranged around the arm at a first axial location along the arm, and the second set of fixation elements can be circumferentially arranged around the arm at a second axial location along the arm, where the second axial location is axially offset or spaced apart from the first axial location (e.g., the second axial location can be proximal to or distal to the first axial location). In some embodiments, the first set of fixation elements are spaced apart or offset circumferentially from the second set of fixation elements. The fixation elements 130 can be symmetrically or asymmetrically distributed about the circumference of the arm, along the length of the arm, and/or between components of the device 100. The number of axially spaced apart fixation elements 130 that are disposed along a length of the arm can be based on the lengths of the fixation elements 130 and/or distances between axially adjacent fixation elements 130. As but one example, if the distal portion 122b of the first arm 122 has a length of about 6 mm and the fixation elements 130 each have a length of about 1 mm, the distal portion 122b can include a maximum of about six fixation elements 130 along its length. In this example, if axially adjacent fixation elements 130 are spaced apart from one another, the distal portion 122b may include two, three, four, or five fixation elements 130 along its length.

In some embodiments, the second end portions 130b of the fixation elements 130 are radially spaced apart from the sidewall 500 to prevent or limit anterior movement of the lead body 104 when the device 100 is implanted. Still, the orientation of one, some, or all of the fixation elements 130 can be opposite of the orientation of the fixation elements 130 shown in FIG. 5 such that the first end portions 130a of such fixation elements 130 are spaced apart from the sidewall 500 while the second end portions 130b of such fixation elements 130 are positioned at the sidewall 500. The second end portion 130b of one or more of the fixation elements 130 can be positioned proximal or distal of the corresponding first end portion 130a of the fixation element 130.

The fixation elements 130 can comprise a portion of the sidewall 500 of the lead and/or can comprise discrete elements secured to the sidewall 500 of the lead. In some embodiments, the fixation elements 130 are formed by cutting the sidewall of the lead and lifting the second end portions 130b of the fixation elements 130 away from the sidewall 500. The fixation elements 130 can be formed by laser cutting (e.g., a UV laser cutting, gas laser cutting, crystal laser cutting, fiber laser cutting, etc.), mechanical cutting (e.g., with a blade), electron beam machining, water jet cutting, or another suitable method. In some embodiments, the lead or one or more portions thereof (e.g., the lead body, the extension portion, etc.) comprises a polymer tube, and the fixation elements 130 are cut from the sidewall of the polymer tube. The polymer can be a thermoplastic material, such as thermoplastic polyurethane. The fixation elements 130 can be bent radially away from the cylindrical plane of the sidewall and heat can be applied to hold the fixation elements 130 in the bent configuration. In some embodiments, the lead is backfilled (e.g., with silicone) to further secure the fixation elements 130.

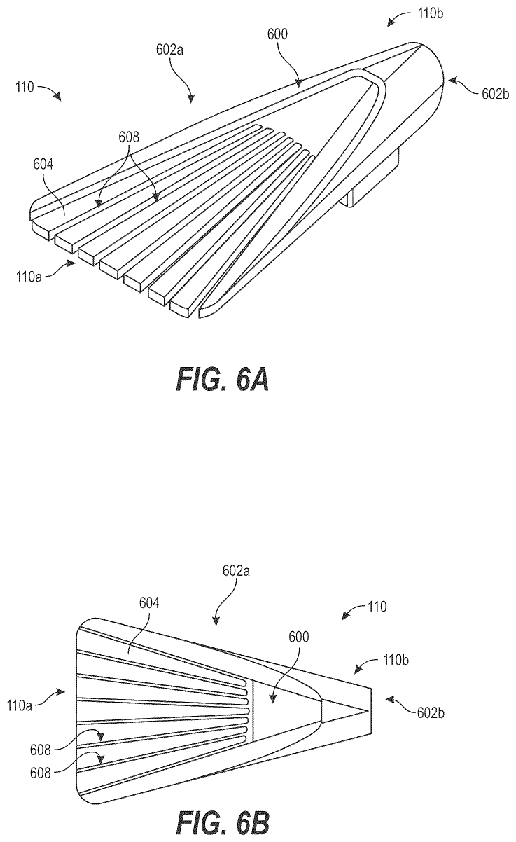

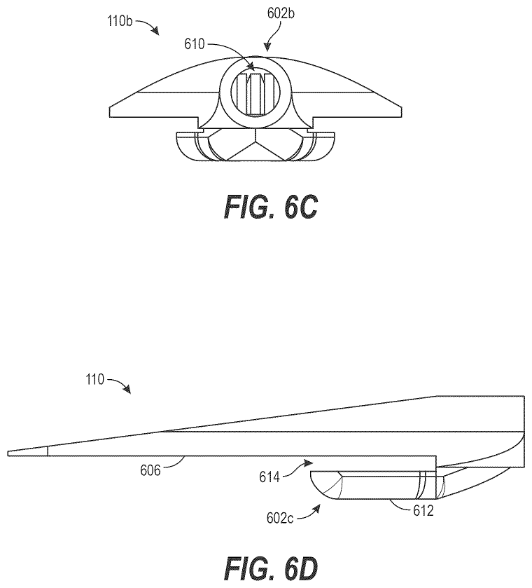

FIGS. 6A-6D are isometric, top, end, and side views, respectively, of the first connector 110 of FIGS. 2B-2D, which can be configured to connect the electronics package 108 to the extension portion 106. The first connector 110 can comprise a proximal portion 110a and a distal portion 110b. A housing 600 of the first connector 110 can include one or more securing portions 602 for securing to another component of the device 100. For example, as shown in FIGS. 6A-6D, the housing 600 can comprise a first securing portion 602a for securing to electrical conductors carried by the extension portion 106, a second securing portion 602b for securing to the extension portion 106, and/or a third securing portion 602c for securing to the electronics package 108. The first securing portion 602a can comprise a first broad surface 604, a second broad surface 606, and a plurality of recesses 608, each of which can be configured to receive an electrical conductor. The first securing portion 602a can be configured to secure to the electrical conductors in a manner that provides strain relief of the electrical conductors to prevent or limit separation of the electrical conductors from the first securing portion 602a and/or damage of the conductors. In some embodiments, the electrical conductors are at least partially soldered, welded, adhered, or otherwise secured to the first securing portion 602a. The second securing portion 602b can comprise a lumen 610 configured to receive the proximal end portion 106a of the extension portion 106. In some embodiments, the proximal end portion 106a of the extension portion 106 can be positioned at least partially in the lumen 610 such that the second securing portion 602b prevents or limits motion of extension portion 106 relative to the electronics package 108. The proximal end portion 106a of the extension portion 106 can be fixedly secured to the first connector 110 by welding, soldering, adhering, gluing, etc. The third securing portion 602c can comprise a projection 612 spaced apart from the second broad surface 606 of the first securing portion 602a to define a gap 614 for receiving the electronics package 108. In some embodiments, the electronics package 108 can be positioned at least partially in the gap 614 such that the projection 612 and/or the second broad surface 606 prevent or limit motion of the electronics package 108 relative to the first connector 110. The electronics package 108 can be fixedly secured to the first connector 110 by welding, soldering, adhering, gluing, etc. The housing 600 can comprise one unitary body or can comprise multiple discrete components secured together after the components have been formed. In some embodiments, the housing 600 comprises a polymeric material and/or is formed by injection molding, additive manufacturing, or another suitable manufacturing technique. The housing 600 can be sufficiently flexible to reduce forces applied to the electrical conductors by motion of the electronics package and/or extension portion 106.

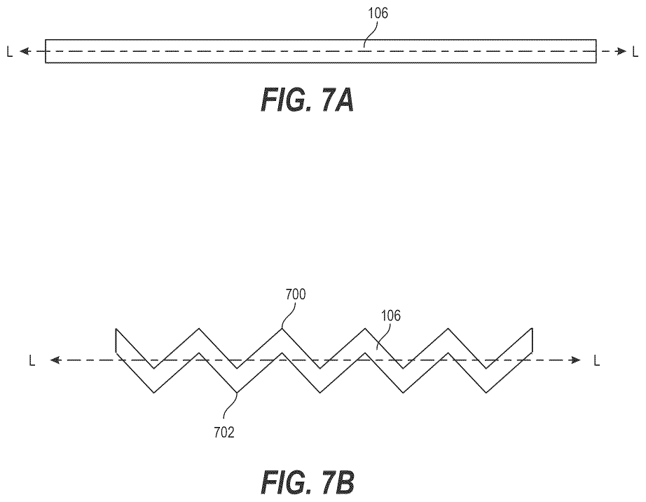

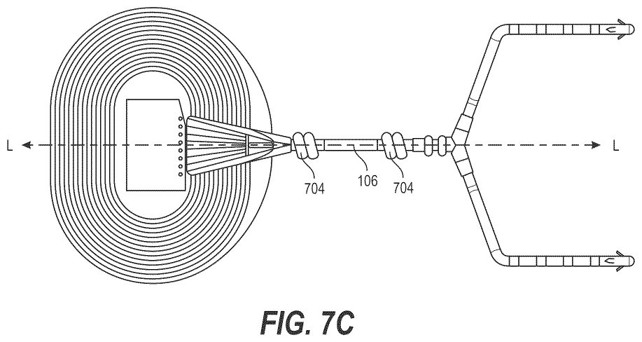

FIGS. 7A-7C show the extension portion 106 of the lead 102 isolated from other components of the device 100. The extension portion 106 can have a number of suitable shapes. For example, the extension portion 106 can be substantially straight along its longitudinal axis L (see FIG. 7A). In some embodiments, the extension portion 106 undulates along its longitudinal axis L between peaks 700 and valleys 702 (see FIG. 7B). As shown in FIG. 7C, the extension portion 106 can comprise one or more helically wound regions 704 in which the extension portion 106 is wound about its longitudinal axis L. A shape, material, and/or other property of the extension portion 106 can be based on a desired functionality of the extension portion 106. For example, the lead body 104 can be configured to be positioned between the genioglossus and the geniohyoid muscles, while the electronics package 108 is configured to be positioned inferior to the mylohyoid. Accordingly, the extension portion 106 can be configured to extend superiorly and wrap anteriorly around the mylohyoid and geniohyoid muscles from the electronics package 108 to the lead body 104. Thus, the extension portion 106 can have a length based on the combined thickness of the mylohyoid and geniohyoid muscles such that, when the conductive elements 114 are located at desired positions in the patient, the extension portion 106 has sufficient length to wrap around the geniohyoid and mylohyoid muscles to position the electronics package 108 at a desired position inferior to the mylohyoid. The extension portion 106 can have a length between about 30 mm and about 90 mm, between about 40 mm and about 80 mm, between about 50 mm and about 70 mm, less than 30 mm, more than 90 mm, about 10 mm, about 20 mm, about 30 mm, about 40 mm, about 50 mm, about 60 mm, about 70 mm, about 80 mm, about 90 mm, or about 100 mm. In some embodiments, a length of the extension portion 106 is based on a distance between a target position of the conductive elements 114 and a target position of the electronics package 108 in a population. For example, the length of the extension portion 106 can be at least partially based on an average thickness of the geniohyoid and mylohyoid muscles in a specific population (e.g., men ages 18 and older, etc.).

In some embodiments, the extension portion 106 can be extendible to accommodate a range of combined geniohyoid and mylohyoid thicknesses. Any of the extension portions 106 disclosed herein (e.g., as shown in FIG. 7A-7C, etc.) can be extendible because of a material property of the extension portion 106 and/or a shape of the extension portion 106 (for example, the undulating and wound shapes shown in FIGS. 7B and 7C, respectively) that facilitates elongation of the extension portion 106 under tensile forces. In some embodiments, the extension portion 106 can have sufficiently high ductility so that the extension portion 106 can be elongated without yielding or failing, as well as having a sufficiently low elasticity such that the extension portion 106 remains in a desired shape after being elongated.

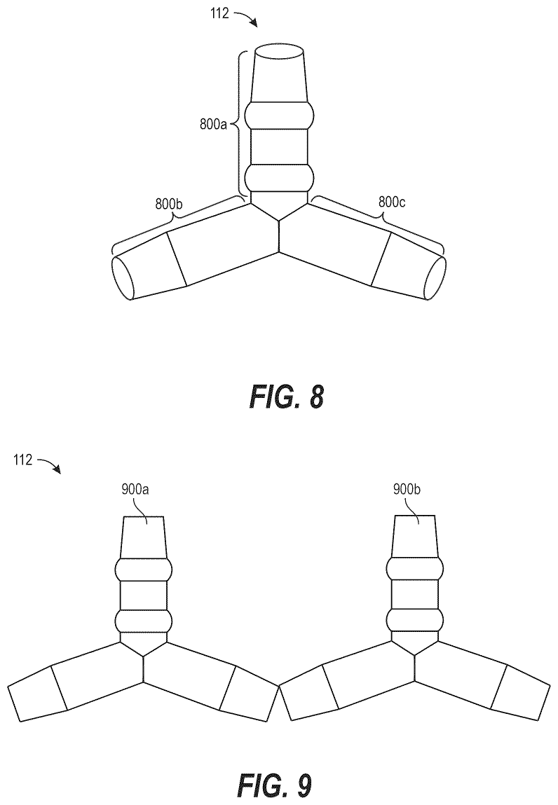

FIG. 8 shows the second connector 112 isolated from other components of the device 100. The second connector 112 can comprise a single unitary body or the second connector 112 can comprise multiple discrete components that are formed separately and later secured to one another. In some embodiments, the second connector 112 comprises three tubular portions: a first tubular portion 800a for securing to the extension portion 106 of the lead 102, a second tubular portion 800b for securing to the first arm 122 of the lead, and a third tubular portion 800c for securing to the second arm 124 of the lead (collectively “tubular portions 800”). The tubular portions 800 can be formed unitarily or as separate components that are later secured together. Each of the tubular portions 800 can define a lumen configured to receive a sidewall of a corresponding component therein. For example, the first tubular portion 800a can be configured to receive the sidewall of the distal end portion 106b of the extension portion 106 therein.

In some embodiments, the second connector 112 can have a clamshell construction in which the second connector 112 is movable between an open configuration and a closed configuration. FIG. 9 illustrates such a second connector 112 in the open configuration. As shown in FIG. 9, the second connector 112 can have a first component 900a and a second component 900b movable relative to the first component 900a. The first and second components 900a, 900b of the clamshell second connector 112 can have substantially the same shape or can have different shapes. In the open configuration, the second component 900b is at least partially separated from the first component 900a. Each of the first and second components 900a, 900b can define an open interior volume when the second connector 112 is in the open configuration. The first component 900a can be connected to the second component 900b at one or more locations in the open configuration. For example, the first component 900a can be connected to the second component 900b by a hinge. In some embodiments, the hinge comprises a thin, flexible piece of material extending between a portion of the first component 900a and a portion of the second component 900b. In some embodiments, the first component 900a can be completely separated from the second component 900b in the open configuration. In the closed configuration, the first and second components 900a, 900b can be brought together and aligned with one another to define an enclosed interior volume of the second connector 112.

This clamshell configuration can facilitate assembly of the lead 102 and tunneling of electrical conductors from the lumen of the lead body 104 into the lumen of the extension portion 106. For example, the second connector 112 can be moved to the open configuration so that the electrical conductors can be laid flat into their respective branches of the first component 900a (or the second component 900b) of the second connector 112. Then the second connector 112 can be moved to the closed configuration by placing the second component 900b over the first component 900a so that the electrical conductors are constrained within their respective branches of the second connector 112. This process may be quicker and easier to execute than threading electrical conductors into tubular portions of the second connector 112. Discrete components of the second connector 112 can be configured to be secured to one another via mechanical fastening (e.g., with mechanical fastener(s), a mechanical interfit such as a friction fit or snap fit, etc.) and/or adhesive. In some embodiments, it may be advantageous to reduce or limit the number of joints between discrete components, which can prevent or limit fluid ingress into the second connector 112 and/or mechanical breakage of the second connector 112.

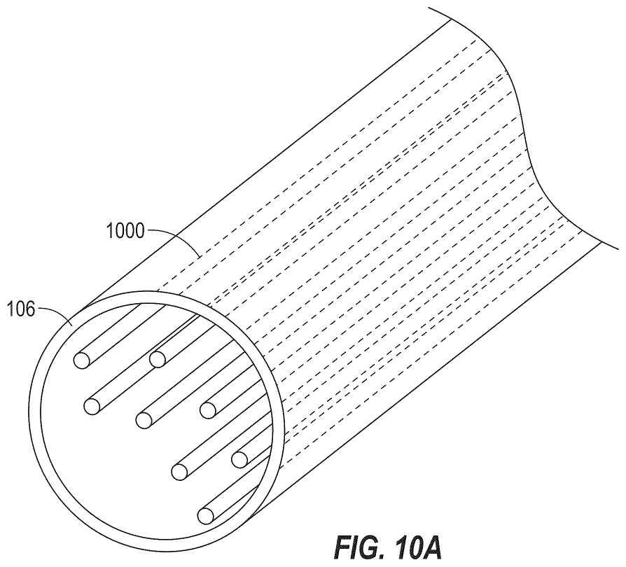

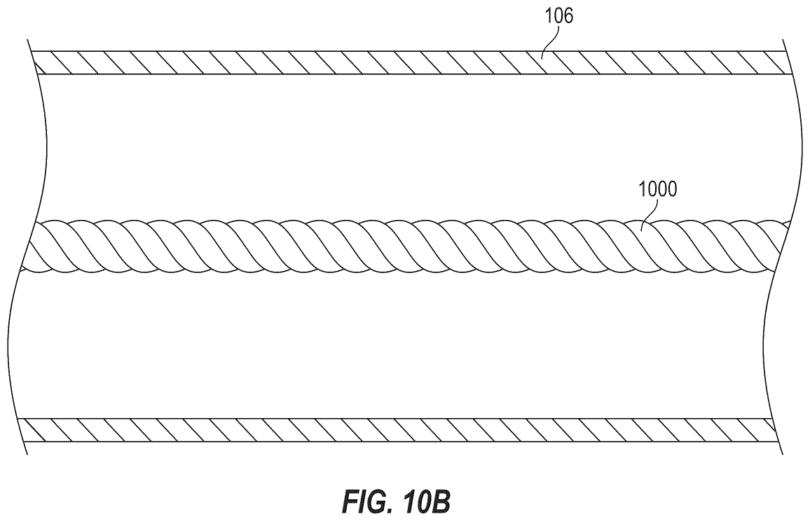

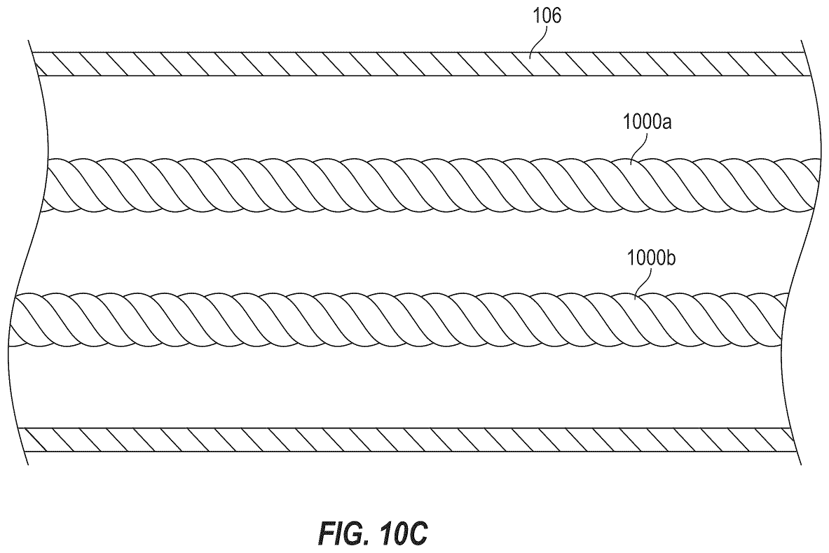

As previously noted, one or more electrical conductors connecting the conductive elements 114 to the electronics package 108 can be carried by the lead 102. The electrical conductors can be positioned on, along, and/or within the lumen of one or more portions of the lead 102 (e.g., the extension portion 106, the first arm 122, the second arm 124, etc.). In some embodiments, for example as shown in FIG. 10A, the electrical conductors 1000 can extend along substantially straight paths through the lumen of the extension portion 106. Additionally or alternatively, the electrical conductors 1000 can extend along substantially straight paths through the lumen of the lead body 104 (e.g., through the lumen of the first arm 122, the lumen of the second arm 124, etc.).

In some embodiments, it can be useful for the electrical conductors to extend along a curved path through the lumen of the extension portion 106. For example, as shown in FIG. 10B, the electrical conductors 1000 can be wound together such that each individual electrical conductor 1000 extends along a helical path through the lumen of the extension portion 106. Another example configuration is shown in FIG. 10C in which a first group of electrical conductors 1000a are wound together, and a second group of electrical conductors 1000b are wound together. The first and second groups of electrical conductors 1000a, 1000b can be positioned adjacent to one another within the lumen of the extension portion 106 (e.g., as shown in FIG. 10C). Additionally or alternatively, the first group of electrical conductors 1000a can be wound about the second group of electrical conductors 1000b, creating a nested coil configuration. In these examples and others, the curved, helical path that each electrical conductor follows provides strain relief so that elongation of the electrical conductor generates less strain in the electrical conductor, thereby improving a fatigue resistance of the electrical conductor.

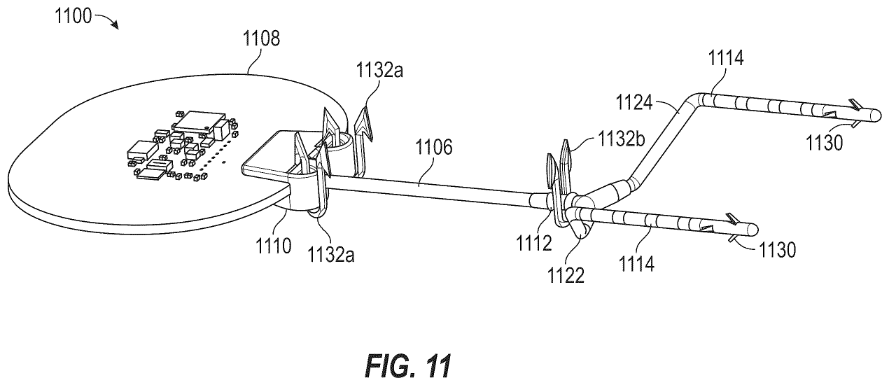

FIG. 11 illustrates an example neuromodulation device 1100 in accordance with several embodiments of the present technology. The features of the device 1100 can be generally similar to the features of the device 100 of FIGS. 2A-10C. Accordingly, like numbers (e.g., fixation elements 1130 versus fixation elements 130) are used to identify similar or identical components in FIGS. 2A-11, and the discussion of the device 1100 of FIG. 11 will be largely limited to those features that differ from the device 100. Additionally, any of the features of the device 1100 can be combined with the features of the device 100.

Similar to device 100, the device 1100 shown in FIG. 11 includes a first arm 1122 and a second arm 1124 each including fixation elements 1130 located distal to conductive elements 1114 of the arm and configured to engage fat surrounding the hypoglossal nerve. Additionally, the device 1100 includes one or more securing elements 1132 configured to secure at least a portion of the device 1100 to the patient's tissue. The securing elements 1132 can comprise a clip, clamp, staple, tine, hook, barb, anchor, suture, or any other suitable element for securing the device 1100 to the patient's tissue. The securing elements 1132 can be bioresorbable or non-bioresorbable. In some embodiments, the securing elements 1132 comprise surgical clips. For example, as shown in FIG. 11, one or more of the securing elements 1132 can comprise a surgical clip with two extensions with a bend between the two extensions. The ends of the extensions can include barbs configured to pierce into tissue and, once engaged, resist separation from the tissue. In some embodiments, the extensions can have equal length such that their ends have generally equal penetrating depth, though in some embodiments the extensions can have varying lengths such that their ends have unequal penetrating depth. Furthermore, in some embodiments, the bend can include a curve, such as a “U”-shaped or “J”-shaped curve.

According to various embodiments, a securing element 1132 is configured to simultaneously engage a portion of the device 100 and tissue surrounding the device when the device is implanted. For example, the extensions and the bend of a securing element 1132 can define a space configured to receive a portion of the device 1100 therein. For example, as shown in FIG. 11, a first retainer 1110 can be configured to retain one or more first securing elements 1132a. The first retainer 1110 can include one or more openings each configured to receive an extension of one of the first securing elements 1132a therein. By retaining the one or more first securing elements 1132a, the first retainer 1110 facilitates coupling a portion of the device 100 to one or more tissues of the patient via the one or more first securing elements 1132a. In some embodiments, the electronics package 1108 or one or more components thereof is configured to retain the first securing element(s) 1132a. For example, a substrate carrying the first antenna and/or at least a portion of the electronics component of the electronics package 1108 can include one or more openings each configured to receive at least a portion of one of the first securing elements 1132a therein. Additionally or alternatively, a coating disposed on the first antenna and/or an enclosure containing at least a portion of the electronics component of the electronics package 1108 can include one or more openings each configured to receive at least a portion of one of the first securing elements 1132a therein. In some embodiments, the openings are formed by removing material from the substrate coating, and/or enclosure. The openings can be defined by forming the substrate, coating, and/or enclosure with open spaces at the openings (e.g., by casting the substrate and/or coating on a mold with positive features defining the openings, forming the substrate and/or coating by additive manufacturing in a specific pattern, etc.). Additionally or alternatively, the openings can be defined by projections extending away from the substrate, coating, and/or enclosure. The openings of the electronics package 1108 configured to receive the first securing elements 1132a therein can be positioned proximate a periphery and/or a central region of the first antenna. In some embodiments, the openings are positioned proximate a central region of the first antenna and/or proximate at least a portion of the electronics component disposed within the central region of the first antenna.

As shown in FIG. 11, a second securing element 1132b can be configured to be positioned around a second retainer 1112. In some embodiments, the second retainer 1112 includes one or more ridges and/or channels to facilitate retaining the second securing element 1132b at a desired location relative to the second retainer 1112. In any case, the securing elements 1132 can be distinct components from the lead 1102 and/or electronics package 1108 such that the device 1100 can be positioned relative to the patient's tissue before securing the device 1100 to the tissue with the securing elements 1132.

The securing elements 1132 can be configured to secure various portions of the device 1100 to different patient tissues. For example, the second securing element 1132b can be configured to secure the second retainer 1112 to the genioglossus muscle of a patient. Additionally or alternatively, the first securing elements 1132a can be configured to secure the first retainer 1110 to the mylohyoid muscle of a patient. In some embodiments, the second securing element 1132b is configured to prevent or limit anterior and/or posterior movement of the device 1100 relative to the genioglossus once implanted. Additionally or alternatively, the second securing element 1132b can be configured to prevent or limit medial movement and/or lateral movement of the device 1100 once implanted. The first securing elements 1132a can be configured to prevent or limit anterior, posterior, medial, and/or lateral movement of the device 1100 relative to the mylohyoid once implanted. In some embodiments, the device 1100 includes at least two first securing elements 1132a to prevent or limit the electronics package 1108 from rotating relative to the mylohyoid, which could occur with only a single first securing element 1132a. For example, the device 1100 can include at least one first securing element 1132a on or adjacent to each of two opposing sides of the electronics package 1108 (e.g., on medial and lateral sides of the electronics package 1108, or of the extension portion 1106), to help prevent or limit rotation of the electronics package 1108 around the axis of the extension portion 1106.

As previously noted, it can be advantageous for a first antenna of a neuromodulation device of the present technology to be flexible and/or conformable so that the first antenna mimics the shape of the patient's anatomy once implanted. However, if the coil of the first antenna includes a material that is corrosive, toxic, carcinogenic, thrombogenic, allergenic, inflammatory, or otherwise not biocompatible, extra precautions should be taken to isolate the coil from the body. For example, copper is susceptible to corrosion in the human body, which can release metallic ions into the body and degrade the performance of a first antenna formed from copper. A coating can be applied to a copper coil to hermetically seal the coil and isolate the coil from the body. However, such a coating is typically thick and/or rigid, which can limit the flexibility and conformability of the first antenna. Moreover, testing and quality requirements for first antennas with non-biocompatible and/or corrosive materials tend to be extensive and may increase the time and costs of development and manufacturing.

To address the aforementioned challenges, some embodiments of the present technology are directed to a first antenna comprising a coil formed from a conductive wire consisting of or encapsulated itself in biocompatible materials (e.g., materials that cause minimal or low thrombogenic, toxic, cancerous, or allergic inflammatory, etc. response when implanted in a patient's body) and/or non-corrosive materials (e.g., materials that do not substantially corrode when implanted in a patient's body). Because such materials may not need to be hermetically sealed, the first antenna can include a flexible, soft, and/or thin coating or housing over the coil, thereby enhancing the flexibility and/or conformability of the first antenna.

A coil of a first antenna of the present technology can be formed from a conductive wire wound into a desired pattern of coil turns. The wire can have mechanical properties (e.g., stiffness, diameter, etc.) that provide a desired flexibility and/or conformability to the first antenna. A coil comprising a wound wire also has certain benefits as compared to a coil comprising traces of conductive material laminated and/or deposited onto a printed circuit board substrate (e.g., polyimide, etc.). Printed circuit board substrates often include multiple layers secured together with adhesive and are susceptible to delamination and degradation due to fluid ingress into the substrate once implanted in the body. Thus, such substrates may be hermetically enclosed to isolate the substrate from the environment of the body. A wire can be carried by a greater variety of substrates than a conductive trace (and also can be standalone with no substrate) and thus, a substrate can be selected that does not require a hermetic enclosure. For example, the substrates carrying the coil wires of the present technology can be biocompatible, hydrophobic, and highly stable within the body. Substrates carrying wires can also be soft and flexible to provide a desired flexibility and/or conformability to the first antenna.

FIG. 13 is a plan view of an electronics package 1308 including a first antenna 1316 and an electronics component 1318 (shown schematically) in accordance with various embodiments of the present technology. The first antenna 1316 can comprise a coil 1334 formed from a wire 1335 having a first end portion 1336 (shown schematically by dashed line) coupled to the electronics component 1318, a second end portion 1338 (shown schematically by dashed line) coupled to the electronics component 1318, and a wound portion 1340 including a plurality of turns 1342 surrounding an opening 1348. In some embodiments, for example as shown in FIG. 13, the electronics component 1318 is positioned within the opening 1348. According to various embodiments, the coil 1334 can be substantially planar such that each of the turns 1342 lies within a two-dimensional plane. Still, in some embodiments, individual turns of the coil 1334 can lie within different two-dimensional planes and/or the coil 1334 can be bent such that certain regions of the coil 1334 are at different elevations to one another.

The wire 1335 can be conductive and configured to carry a current. The wire 1335 can comprise a biocompatible material that, when implanted in a submental region of a patient, does not substantially cause a thrombogenic, toxic, cancerous, or allergic inflammatory response. In various embodiments, the wire 1335 can comprise a material that is configured to experience little to no corrosion when implanted in the submental region. The wire 1335 can comprise or consist of, for example, gold, graphene, platinum, titanium, and/or alloys thereof. The wire 1335 can comprise a single strand or a plurality of strands. Additionally or alternatively, the wire 1335 can comprise a single material or multiple materials. In various embodiments, the wire 1335 can be include a first, core material and a second material disposed on the first material. In some embodiments, the first material is not biocompatible and/or non-corrosive but the second material is biocompatible and/or non-corrosive and isolates the first material from the environment. The second material can be conductive. The wire 1335 can be formed with the first and second materials by plating, sputtering, drawing, or any other suitable method.

The wire 1335 can have surface treatments (e.g., plasma treatments, surface roughening, etc.) to facilitate coupling of the wire 1335 to a substrate and/or a coating. In some embodiments, the wire 1335 can be insulated in a non-conductive material along some or all of the length of the wire 1335. The non-conductive insulating material can comprise, for example, polyimide, PTFE, urethanes, silicones, Parylene, combinations thereof, or other suitable materials. The non-conductive insulation wire may be applied before the coiling process or may be applied after coiling the wire in the desired geometry. In various embodiments, the wire 1335 can be insulated such that, when implanted in the body, a resonant frequency of the first antenna 1316 does not substantially change. In contrast, when a first antenna comprising a conductive trace carried on a polyimide is implanted in the body, the resonant frequency of the first antenna may change, which may require tuning of the resonant circuit of the first antenna and/or a second antenna which the first antenna is intended to inductively couple to.

The wire 1335 can comprise an elongate member having any suitable shape and/or dimensions. In some embodiments, the wire 1335 has a cross-sectional shape that is round, rectangular, triangular, polygonal, or irregular. The wire 1335 can comprise material that has been extruded, drawn, cast, deposited, cut, stamped, machined, rolled, or otherwise formed into an elongate member that can then be shaped to form the desired turns 1342 of the wound portion 1340 of the coil 1334.

The wire 1335 can have a cross-sectional shape with a constant diameter along a length of the wire 1335 or the wire 1335 can have a cross-sectional shape with a diameter that varies along the length of the wire 1335. In some embodiments, the diameter of the cross-sectional shape of the wire 1335 can be based at least in part on a desired power harvesting performance of the first antenna 1316. For example, due to its greater outer surface area, a wire 1335 with a larger diameter has higher electrical conductance than a wire 1335 with a smaller diameter, which can facilitate greater power harvesting be reducing resistive and inductive (e.g., self-inductive) losses as current is induced through the coil 1334. However, for a coil 1334 with a fixed area and a fixed number of turns, adjacent turns 1342 formed from a wire 1335 with a larger diameter will be closer together and may generate greater parasitic capacitance than a wire 1335 with a smaller diameter. Thus, the diameter of the wire 1335 may be selected to balance electrical conductance and parasitic capacitance. Compared with a wire 1335 having a smaller diameter, a wire 1335 with a larger diameter will also be stiffer and thus the first antenna 1316 will be less flexible. Such a first antenna 1316 may, however, advantageously be configured to retain a shape corresponding to the shape of the submental region within which the first antenna 1316 is configured to be placed. Accordingly, the diameter of the wire 1335 may be selected to balance flexibility and conformability of the first antenna 1316. In various embodiments, the diameter of the wire 1335 can be between about 0.15 mm and about 0.30 mm, about 0.15 mm, about 0.20 mm, about 0.25 mm, or about 0.30 mm.

As shown in FIG. 13, the coil 1334 can have a width W measured in a first dimension and a length L measured in a second dimension. According to various embodiments, the width W can be larger than the length L. The coil 1334 can have a shape that is generally oblong and/or elongated (e.g., obround, stadium, elliptical, ovular, rectangular, etc.). The coil 1334 can have a shape and/or dimensions based on a desired anatomical placement of the coil 1334. For example, as previously described herein, the coil 1334 can be configured to be implanted in a submental region bound superiorly by the mylohyoid and inferiorly by the platysma. The submental region can also be bound in the sagittal plane anteriorly by the mentum and posteriorly by the hyoid. The coil 1334 can be configured to be implanted in the submental region with the length L of the coil 1334 aligned with the sagittal plane and thus, the dimension of the length L can be based on a distance between the mentum and the hyoid for a particular patient and/or a population of patients. Moreover, the dimension of the length L can be based on the distance between the mentum and the hyoid in one or more postures. For example, the distance between the mentum and the hyoid with the neck in a neutral posture can be between about 35 mm and about 55 mm in a population of patients. However, when the neck is flexed, the distance between the mentum and the hyoid can decrease about 30% to about 40%. Thus, in some embodiments the length L is selected to prevent or limit contact between the coil 1334 and the hyoid or the mentum during neck flexion. For example, the length L can be no greater than about 40 mm, no greater than about 35 mm, no greater than about 30 mm, no greater than about 25 mm, or no greater than about 20 mm. For a given device to accommodate patients with a range of mentum to hyoid distances, the length L can be based on a minimum expected hyoid to mentum distance in a population of patients. In these embodiments, and others, the length L can be about 25 mm, about 24 mm, about 23 mm, about 22 mm, about 21 mm, about 20 mm, about 19 mm, about 18 mm, or about 17 mm.

Additionally or alternatively, the coil 1334 can have a shape based on a desired power to be harvested by the coil 1334. For example, if the length L of the coil 1334 is decreased relative to the diameter of a circular coil based on a desired fit of the first antenna 1316 within an anatomical region, the width W of the coil 1334 can be increased to maintain a desired surface area of conductive material within the coil 1334, which influences how much power the coil 1334 can harvest within a given electromagnetic field. Additionally or alternatively, the width W of the coil 1334 can be selected to enable the placement of the electronics component 1318, securing element, or other component(s) within the opening 1348. The width W of the coil 1334 may also be limited by anatomical constraints. For example, the width W of the coil can be selected to prevent or limit contact between the coil 1334 and the mandible when implanted in a submental region of a patient. Thus, the oblong shape of the coil 1334 facilitates both anatomical placement and power harvesting. In some embodiments, the width W of the coil 1334 can be between about 35 mm and about 50 mm, between about 40 mm and about 50 mm, between about 45 mm and about 50 mm, about 35 mm, about 40 mm, about 45 mm, or about 50 mm. In various embodiments, a ratio of the width W of the coil 1334 to the length L of the coil 1334 can be about 1.5 to 1, about 2 to 1, about 2.5 to 1, or about 3 to 1.

Referring still to FIG. 13, each of the turns 1342 can extend from a first end 1342a to a second end 1342b. The first ends 1342a of the turns 1342 can be generally aligned with one another in a radial direction, and similarly the second ends 1342b of the turns 1342 can be generally aligned with one another in a radial direction (e.g., the first ends 1342a and/or the second ends 1342b can be aligned along a ray extending generally from a central region of the coil toward the outermost coil turn 1342). In the example shown in FIG. 13, the first ends 1342a of the turns 1342 can be substantially aligned with one another at any given location along the width W of the coil 1334 and located at different locations along the length L of the coil 1334. Likewise, the second ends 1342b of the turns 1342 can be substantially aligned with one another at any given location along the width W of the coil 1334 and located at different locations along the length L of the coil 1334. As yet another example, the first ends 1342a of the turns 1342 can be substantially aligned with one another at any given location along the length L of the coil 1334 and located at different locations along the width W of the coil 1334 and the second ends 1342b of the turns 1342 can be substantially aligned with one another at any given location along the length L of the coil 1334 and/or located at different locations along the width W of the coil 1334. It should be understood that the first ends 1342a and the second ends 1342b can each be positioned at any suitable perimetral location around the coil 1334.

As shown in FIG. 13, the first end 1342a of a given turn 1342 (excluding the innermost turn 1342) can be positioned at, continuous with, and/or electrically coupled to the second end 1342b of a preceding turn 1342. Thus, the coil 1334 can comprise a continuous coil with sequential turns 1342 electrically coupled to their preceding turns 1342. In other words, the turns 1342 are spiral turns that form a spiral wound portion 1340 of the coil 1334. One, some, or all of the turns 1342 can form a substantially complete loop with the second end 1342b of the turn 1342 aligned with or proximate to the first end 1342a of the turn 1342 along the width W of the coil 1334. In some embodiments, the first end 1342a of a given turn 1342 can be spaced apart from the second end 1342b of the preceding turn 1342 along the length L and/or width W of the coil 1334.

In various embodiments, one or more of the turns 1342 can include one or more straight portions 1344 and/or one or more curved portions 1346. In some embodiments, the straight portions 1344 and curved portions 1346 alternate along the length of the turn 1342. For example, FIG. 13 illustrates each of the turns 1342 having two straight portions 1344 (only labeled on the innermost turn 1342 for clarity of illustration) opposite one another along the length L of the coil 1334 and two curved portions 1346 (only labeled on the innermost turn 1342 for clarity of illustration) opposite one another along the width W of the coil 1334. Thus, the turns 1342 shown in FIG. 13 define the obround shape of the coil 1334. In embodiments in which the coil 1334 has an elliptical or ovular shape, for example, one or more of the turns 1342 can include only curved portions 1346. Conversely, in embodiments in which the coil 1334 has a rectangular shape, for example, one or more of the turns 1342 can include only straight portions 1344.

Although FIG. 13 illustrates the wound portion 1340 including seven turns 1342, the wound portion 1340 can include any suitable number of turns 1342. For example, the wound portion 1340 can include four turns 1342, five turns 1342, six turns 1342, seven turns 1342, or eight turns 1342. The number of turns 1342 can be based at least in part on a desired power harvesting performance of the first antenna 1316. Increasing the number of turns 1342 will increase the surface area of the conductive material of the coil 1334, thereby increasing the amount of power the coil 1334 can harvest from a given electromagnetic field. However, for a given coil 1334 width W and length L, a wound portion 1340 with a greater number of turns 1342 will have a smaller pitch with adjacent turns 1342 positioned closer together, which can increase the parasitic capacitance of the coil 1334. Thus, the wound portion 1340 can include a number of turns 1342 that balances conductive surface area and parasitic capacitance to enhance the power harvesting capacity of the first antenna 1316.

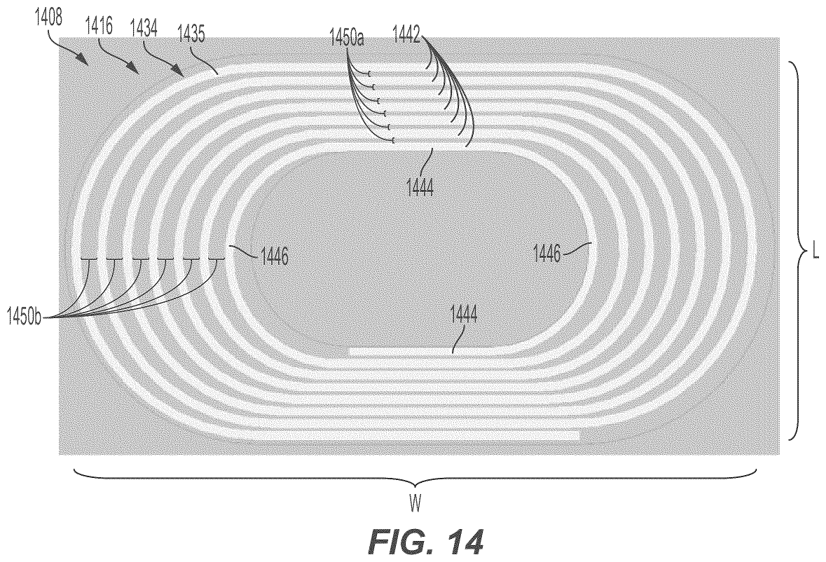

The coil 1334 can have a constant pitch or a variable pitch. For example, FIG. 13 illustrates the coil 1334 having a constant pitch with a distance 1350 between adjacent turns 1342 being substantially constant at all regions of the coil 1334. As another example, FIG. 14 is a plan view of an electronics package 1408 comprising a first antenna 1416 including a coil 1434 formed from a wire 1435 and having a variable pitch. The electronics package 1408, first antenna 1416, coil 1434, and wire 1435 of FIG. 14 can be similar to the electronics package 1308, first antenna 1316, coil 1334, and wire 1335 respectively, of FIG. 13, except as detailed below.

As shown in FIG. 14, in some embodiments the distance 1450a between the straight portions 1444 of adjacent turns 1442 along the length L of the coil 1434 can be substantially constant. Additionally or alternatively, the distance 1450b between the curved portions 1446 of adjacent turns 1442 can differ from the distance 1450a between the straight portions 1444 of adjacent turns 1442 and/or can vary along certain dimensions of the coil 1434. For example, the distance 1450b between the curved portions 1446 can be greater than the distance 1450a between the straight portions 1444. In some embodiments, the distance 1450b between adjacent curved portions 1446 can increase along the width W of the coil 1434. For example, the distance 1450b between the curved portion 1446 of a first innermost turn 1442 and the curved portion 1446 of a second innermost turn 1442 adjacent to the first innermost turn 1442 can be smaller than the distance 1450b between the curved portion 1446 of the second innermost turn 1442 and the curved portion 1446 of a third innermost turn 1442 adjacent to the second innermost turn 1442.

The pitch(es) of the coils disclosed herein can be selected based on a desired amount of power to be harvested by the coil in a given electromagnetic field. A coil with a smaller pitch can have a greater surface area of conductive material within a set of given coil dimensions but may have greater parasitic capacitance between adjacent turns because the adjacent turns are closer together. Varying the pitch of the coil at certain regions of the coil, for example as described with reference to the coil 1434 shown in FIG. 14, can provide a balance between conductive surface area and parasitic capacitance, enabling the coil 1434 to harvest more power from a given electromagnetic field than a coil with constant pitch.

A first antenna of the present technology can comprise a single coil, for example as shown in FIGS. 13 and 14. In some embodiments, a first antenna can comprise multiple coils to increase the surface area of conductive material of the first antenna and thereby the power that the first antenna can harvest from a given electromagnetic field relative to a first antenna with a single coil. As described elsewhere herein, the multiple coils can be electrically connected in parallel to increase the conductive surface area of the first antenna without significantly increasing the series resistance of the first antenna. Still, in some embodiments the length, width, pitch, wire diameter, number of turns, and/or other parameters of a single coil can be selected to provide sufficient power harvesting capability for a given application. Moreover, it may be simpler and/or less expensive to manufacture a first antenna with a single coil instead of multiple coils, particularly if expensive materials such as gold are used for the coil wire.

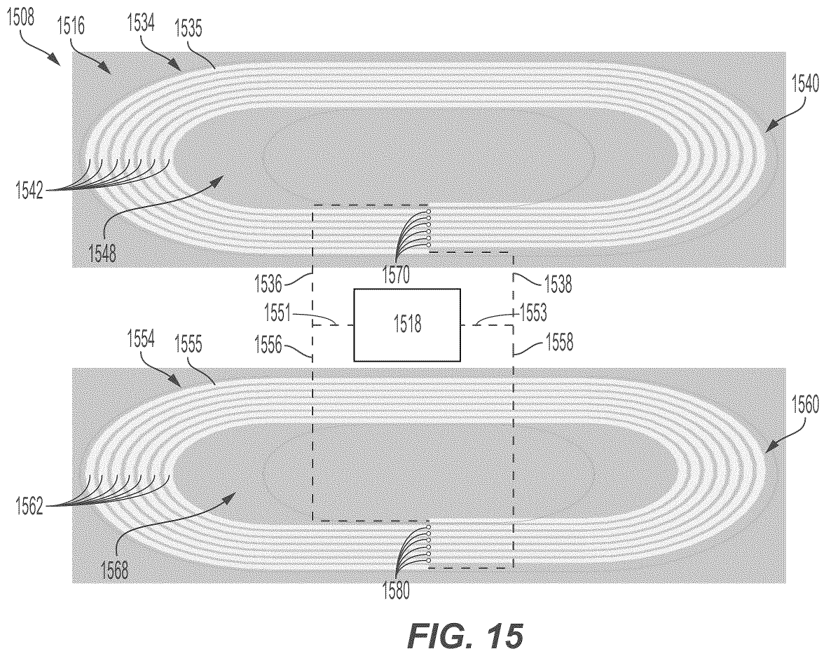

FIG. 15 is an exploded perspective view of an electronics package 1508 comprising a first antenna 1516 including a first coil 1534 and a second coil 1554, each of which is electrically coupled to an electronics component 1518. The first coil 1534 and/or the second coil 1554 can have similar features as the coil 1334 shown in FIG. 13 and/or the coil 1434 shown in FIG. 14 and/or the coil 1334 shown in FIG. 13 and/or the coil 1434 shown in FIG. 14 can have similar features as the first coil 1534 and/or the second coil 1554 shown in FIG. 15.

In various embodiments, the first and second coils 1534, 1554 can be spaced apart from one another along a thickness of the first antenna 1516. For example, as described in greater detail below, the first and second coils 1534, 1554 can be carried by a substrate (not shown in FIG. 15) and spaced apart along a thickness of the substrate. Still, in some embodiments, the first and second coils 1534, 1554 are not carried by a substrate. In these embodiments, and others, the first coil 1534 can be positioned directly on top of the second coil 1554.

The first and second coils 1534, 1554 can be similar to the coils described elsewhere herein (e.g., coil 1334, coil 1434, etc.). For example, the first coil 1534 can be formed from a first wire 1535 having a first end portion 1536 (shown schematically by dashed line) coupled to the electronics component 1518, a second end portion 1538 (shown schematically by dashed line) coupled to the electronics component 1518, and a first wound portion 1540 including a plurality of first turns 1542 surrounding a first opening 1548. Likewise, the second coil 1554 can be formed from a second wire 1555 having a first end portion 1556 (shown schematically by dashed line) coupled to the electronics component 1518, a second end portion 1558 (shown schematically by dashed line) coupled to the electronics component 1518, and a second wound portion 1560 including a plurality of second turns 1642 surrounding a second opening 1568. In some embodiments, the electronics component 1518 is positioned within the first opening 1548 and/or the second opening 1568.

In some embodiments, the second wire 1555 can be continuous with the first wire 1535 such that the first and second coils 1534, 1554 are formed from a single, continuous wire. For example, a single wire can be wound along the lengths and widths of the first and second coils 1534, 1554 to form the first and second turns 1542, 1562 and the wire can be wound along the thickness of the first antenna 1516 to form electrical connectors between corresponding turns of the first and second coils 1534, 1554.

According to some embodiments, the diameter of the first and second wires 1535, 1555 can be smaller than the diameter of a wire in a first antenna comprising a single coil. For example, a first antenna comprising a single coil can be formed from a wire having a diameter of about 0.25 mm while the first and second wires 1535, 1555 can each have a diameter of about 0.18 mm.

The first coil 1534 (or one or more components thereof) can have the same structure as the second coil 1554 (or the corresponding component thereof). For example, the first wound portion 1540 can have the same shape and/or dimensions (e.g., length, width, etc.) as the second wound portion 1560. In some embodiments, the first turns 1542 of the first wound portion 1540 can be aligned with the second turns 1562 of the second wound portion 1560 along the lengths and widths of the first and second coils 1534, 1554. For example, the entire length of each of the first turns 1542 can be aligned with the entire length of a respective one of the second turns 1562. The first ends of the first turns 1542 can be aligned with the first ends of the second turns 1562 and/or the second ends of the first turns 1542 can be aligned with the second ends of the second turns 1562. In various embodiments, the pitch(es) of the first coil 1534 can be the same as the pitch(es) of the second coil 1554.

As schematically depicted in FIG. 15, the first end portion 1536 of the first wire 1535 can be coupled to the first end portion 1556 of the second wire 1555 to form a first cable 1551 that couples to the electronics component 1518 and/or the second end portion 1538 of the first wire 1535 can be coupled to the second end portion 1558 of the second wire 1555 to form a second cable 1553 that couples to the electronics component 1518. As discussed in greater detail below, bundling the wire ends into the first and second cables 1551, 1553 can facilitate maintenance of a hermetic enclosure of the electronics component 1518 by reducing the required number of openings into the coating or housing of the electronics component 1518. Moreover, electrically coupling the first end portions 1536, 1556 to one another and electrically coupling the second end portions 1538, 1558 to one another couples the first coil 1534 to the second coil 1554 in parallel. As previously described herein, the first and second coils 1534, 1554 can be connected in parallel to reduce parasitic capacitance of the first antenna 1516.

Moreover, in some embodiments, each first turn 1542 can be electrically connected in parallel to a corresponding second turn 1562 to reduce the parasitic capacitance of the first antenna 1516. The first turns 1542 can be connected to corresponding second turns 1562 by electrical connectors extending between the first and second turns 1542, 1562. An electrical connector of the present technology can comprise a conductive material contacting both a first turn 1542 at a first electrical contact 1570 and a corresponding second turn 1562 at a corresponding second electrical contact 1580. According to various embodiments, the first electrical contact 1570 and the second electrical contact 1580 comprise locations along the first turn 1542 and the second turn 1562, respectively, at which the electrical connector contacts the respective turn and/or the turns contact one another. The first and second electrical contacts 1570, 1580 may or may not comprise discrete electrical components. In some embodiments, the electrical connector comprises solder and the connection between corresponding first and second turns 1542, 1562 at the first and second electrical contacts 1570, 1580 is formed by traditional soldering, laser soldering, or another suitable soldering process. If the first wire 1535 and/or the second wire 1555 is insulated, the insulating material can be removed at the location of the first and second electrical contacts 1570, 1580 to not impede the electrical connection between the corresponding first and second turns 1542, 1562. In some embodiments, the electrical connector can be formed by welding the corresponding first and second turns 1542, 1562 together at the first and second electrical contacts 1570, 1580, crimping the corresponding first and second turns 1542, 1562 together at the first and second electrical contacts 1570, 1580, positioning a conductive material between the corresponding first and second turns 1542, 1562 together at the first and second electrical contacts 1570, 1580, or another suitable method.

The first coil 1534 can include at least one first electrical contact 1570 per first turn 1542 and/or the second coil 1554 can include at least one second electrical contact 1580 per second turn 1562. As previously noted, the first end portion 1536 of the first wire 1535 can be electrically coupled to the first end portion 1556 of the second wire 1555, effectively coupling the first end of the innermost first turn 1542 to the first end of the innermost second turn 1562. Additionally or alternatively, the second end portion 1538 of the first wire 1535 can be electrically coupled to the second end portion 1558 of the second wire 1555, effectively coupling the second end of the outermost first turn 1542 to the second end of the outermost second turn 1562. Thus, in some embodiments, the first and second ends of each first turn 1542 can be electrically coupled to the first and second ends of a corresponding second turn 1562. If the first wire 1535 includes multiple strands, the first coil 1534 can include at least one first electrical contact 1570 per strand in the first wire 1535 per first turn 1542. If the second wire 1555 includes multiple strands, the second coil 1554 can include at least one second electrical contact 1580 per strand in the second wire 1555 per second turn 1562.

As shown in FIG. 15, in some embodiments the first electrical contact 1570 of a given first turn 1542 is aligned with the second electrical contact 1580 of a corresponding second turn 1562 at a given location along the lengths and widths of the first and second coils 1534, 1554. An electrical connector extending between the corresponding first and second electrical contacts 1570, 1580 can therefore extend substantially straight along the thickness of the first antenna 1516. In some instances, this electrical connector orientation can be advantageous, as if the corresponding first and second electrical contacts 1570, 1580 were not aligned and the electrical connector instead extended laterally along the lengths and/or widths of the coils through the thickness of the first antenna 1516 (e.g., through the antenna substrate in a manner not orthogonal to the plane of the coils), the electrical connector could undesirably modify (e.g., increase or decrease) the impedance of the first antenna 1516.

The first and second electrical contacts 1570, 1580 can be disposed at any suitable perimetral location around the first and second coils. According to various embodiments, the first electrical contacts 1570 can be substantially aligned with the first end of a first turn 1542 and the second end of the preceding first turn 1542 and/or the second electrical contacts 1580 can be substantially aligned with the first end of a second turn 1562 and the second end of the preceding second turn 1562. In the example shown in FIG. 15, the first electrical contacts 1570 can be substantially aligned with one another at any given location along the width of the first coil 1534 and located at different locations along the length of the first coil 1534. Likewise, the second electrical contacts 1580 can be substantially aligned with one another at any given location along the width of the second coil 1554 and located at different locations along the length of the second coil 1554. As yet another example, the first electrical contacts 1570 can be substantially aligned with one another at any given location along the length of the first coil 1534 and located at different locations along the width of the first coil 1534 and the second electrical contacts 1580 can be substantially aligned with one another at any given location along the length of the second coil 1554 and located at different locations along the width of the second coil 1534. In some embodiments, the first and/or second electrical contacts 1570, 1580 can be generally aligned with one another in a radial direction (e.g., the first electrical contacts 1570 and/or the second electrical contacts 1580 can be aligned along a ray extending generally from a central region of the coil toward the outermost coil turn).

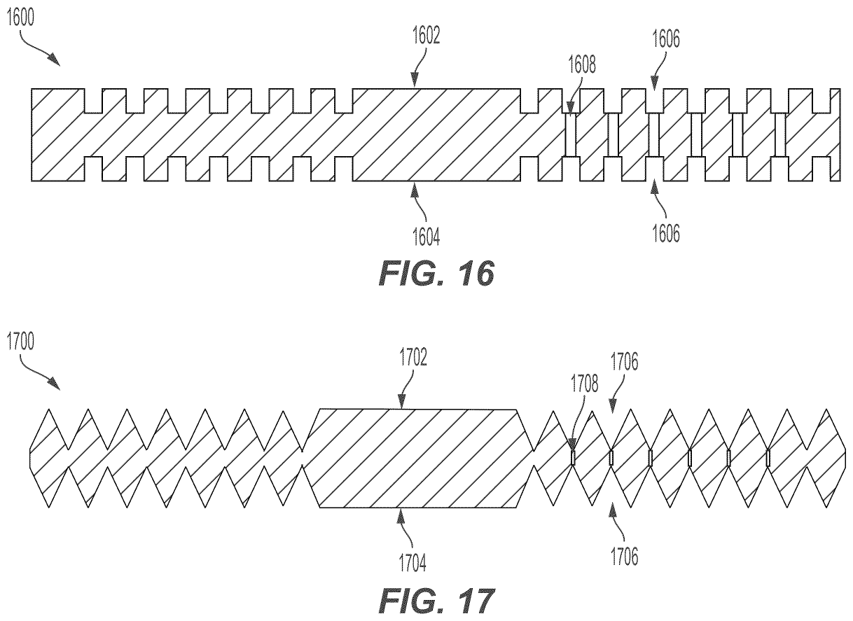

Any of the first antennas disclosed herein can include a substrate carrying one or more coils. The substrate can comprise a single substrate or multiple substrates secured to one another via adhesive, welding, heat, etc. According to various embodiments, the substrate can include features configured to facilitate winding and/or retention of a wire in a desired pattern of turns, for example as shown and described with reference to FIGS. 16-18.

FIG. 16 is a side view of a substrate 1600 configured in accordance with various embodiments of the present technology. The substrate 1600 can have a first broad side 1602 and a second broad side 1604 opposite the first broad side 1602 along a thickness of the substrate 1600. The first broad side 1602 and/or the second broad side 1604 can define one or more grooves 1606 configured to receive a wire therein to facilitate (e.g., guide) winding and/or retention of the wire in a desired pattern of turns. In some embodiments, each of the first broad side 1602 and/or the second broad side 1604 can include a single groove 1606 shaped as a single continuous spiral groove, or can include multiple discontinuous grooves that are collectively laid in a spiral pattern (e.g., spaced-apart segments of a single spiral groove). In embodiments in which the first antenna comprises a single coil, the broad side carrying the coil can define the grooves 1606. In embodiments in which the first antenna comprises two coils, each of the first and second broad sides 1602, 1604 can define the grooves 1606 and be configured to carry the first and second coils, respectively, therein. A configuration of the grooves 1606 within the substrate 1600 can be based on the desired pattern of turns of the respective coil. For example, the grooves 1606 at the first broad side 1602 can be aligned with the grooves 1606 at the second broad side 1604 so that the first turns of a first coil carried by the first broad side 1602 are aligned with the second turns of a second coil carried by the second broad side 1604.

In some embodiments, the substrate 1600 defines one or more openings 1608 extending through a thickness of the substrate 1600 and configured to receive an electrical connector (e.g., for coupling to electrical contacts on each of the first and second coils). For example, the opening 1608 can be configured to receive solder, a wire, or another conductive material that electrically connects a first turn retained by a groove 1606 at the first broad side 1602 to a corresponding second turn retained by a groove 1606 at the second broad side 1604. Thus, in some embodiments, for example as shown in FIG. 16, the openings 1608 can extend between corresponding grooves 1606 through a thickness of the substrate 1600. Still, in some embodiments the substrate 1600 does not include grooves 1606 but does include openings 1608 corresponding to desired locations of electrical contacts on the first and second coils. In embodiments in which a single coil is carried by the substrate 1600, the substrate 1600 may not include the openings 1608 or may include the openings 1608 to facilitate coupling the coil to other electrical components.

According to various embodiments, the substrate 1600 can be formed from a flat sheet of material. The grooves 1606 can be defined by protrusions extending away from a flat surface of the sheet and/or by recesses cut or otherwise formed in the sheet. Still, in some embodiments the substrate 1600 can be formed into a contoured surface defining the grooves 1606 by any suitable process including, for example, additive manufacturing, casting, molding, and/or others. The grooves 1606 have dimensions and/or shapes corresponding to the dimensions and/or shapes of a wire to be retained within the grooves 1606.

Although the grooves 1606 are shown having a rectangular cross-sectional shape in FIG. 16, the grooves 1606 can have any suitable cross-sectional shape, including, for example, round, polygonal, or irregular. FIG. 17, for example, illustrates a substrate 1700 defining grooves 1706 with triangular cross-sectional shapes. The features of substrate 1700 can be combined with or substituted for the features of substrate 1600 and/or the features of substrate 1600 can be combined with or substituted for the features of substrate 1700. For example, the substrate 1700 can comprise first and second broad surfaces 1702, 1704 defining the grooves 1706. Additionally or alternatively, the substrate 1700 can define one or more openings 1708 configured to retain electrical connectors configured to contact the corresponding turns of first and second coils carried by the substrate 1700.

In some embodiments, for example as shown in FIG. 17, the first broad surface 1702 and/or the second broad surface 1704 can undulate to define a plurality of peaks and a plurality of valleys between the peaks. The valleys can define the bottoms of the grooves 1706. In various embodiments, valleys of the first broad surface 1702 can be aligned with valleys of the second broad surface 1704 so that coil turns retained in the valleys are aligned. Although FIG. 17 illustrates the first and second broad surfaces 1702, 1704 as having sharp peaks and valleys, in some embodiments the first and second broad surfaces 1702, 1704 have smooth peaks and/or valleys. In some embodiments, the grooves 1706 have dimensions and/or shapes corresponding to the dimensions and/or shapes of the wire to be retained within the grooves 1706.

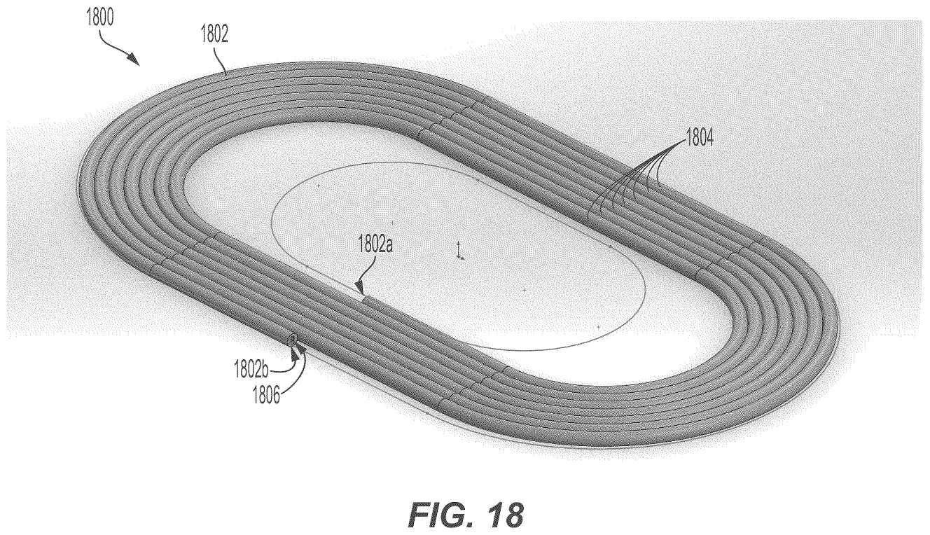

In some embodiments, for example as shown in FIG. 18, the substrate 1800 can comprise an elongate shaft 1802 forming a plurality of turns 1804 as the elongate shaft 1802 extends from a first end 1802a to a second end 1802b. The elongate shaft 1802 defines a lumen 1806 extending along its length and extending through the first and second ends 1802a, 1802b. The lumen 1806 can be configured to receive a wire of the present technology such that the wire assumes the shape of the elongate shaft 1802 and the turns 1804. The turns 1804 of the elongate shaft 1802 can correspond to a desired pattern of turns of the wound portion of a coil so that, when the wire is positioned within the lumen 1806, the wire forms the desired pattern of turns. In some embodiments, a diameter of the elongate shaft 1802 can define a minimum pitch of the coil.

The elongate shaft 1802 can be a tubing (e.g., polymeric tubing, etc.). The elongate shaft 1802 can comprise the same material(s) as the substrate material(s) disclosed herein. For example, in some embodiments, the elongate shaft 1802 comprises a urethane and/or a silicone. The elongate shaft 1802 can comprise a material that is biocompatible, hydrophobic, and/or non-corrosive. In some embodiments, the elongate shaft 1802 is soft and/or flexible. The elongate shaft 1802 can be configured to be plastically deformed to retain a shape forming the turns 1804. Additionally or alternatively, the elongate shaft 1802 can be configured to be heat set into a shape forming the turns 1804. The substrate 1800 can be configured to bend or deform to conform to the patient's anatomy while maintaining the turns 1804 in a predetermined arrangement (e.g., pitch, planar shape, etc.).

In embodiments in which the antenna includes multiple coils, each coil can be carried by a substrate such as that shown in FIG. 18. The elongate shaft 1802 can include one or more openings along its length at the locations of the electrical contacts such that the wire can be electrically coupled to the wire of another coil via the opening(s). Such electrical contacts can have the same features as the electrical contacts described with reference to FIG. 15.

Any of the substrates disclosed herein can comprise a single substrate or multiple substrates secured to one another via adhesive, welding, heat, etc. The substrate can be soft and/or flexible to enhance the flexibility of the first antenna. As previously noted, it may be advantageous for the substrate to be biocompatible, hydrophobic, and/or non-corrosive so that the substrate can be placed within the body without being hermetically enclosed. In various embodiments, the substrate can comprise a polymer such as, but not limited to, a urethane (including polyurethanes) and/or a silicone.

A first antenna of the present technology can comprise a coating disposed on and/or covering the coil(s) and/or substrate(s) of the first antenna. As previously described, a coil comprising a conductive wire that is biocompatible and/or non-corrosive and/or a substrate that is biocompatible, hydrophobic, and/or non-corrosive can enable the use of a coating that is soft, flexible, and/or thin because the wire and/or substrate do not have to be hermetically enclosed. Although it is not necessary for the coils and/or substrates of the present technology to be hermetically enclosed, in some embodiments a coil and/or substrate within the scope of the present technology can be hermetically enclosed (e.g., sealed).

According to various embodiments, the first antenna can include a coating disposed on the coil and/or substrate comprising a polymer such as, but not limited to, a urethane (including polyurethanes) and/or a silicone. Urethanes may be advantageous as coating materials as urethanes can be highly resistant to enzymatic breakdown in the body and/or in contact with blood. In some embodiments, the coating material can be selected based on a desired amount of adhesion between the first antenna and patient tissues. For example, silicones can adhere to patient tissues to a greater degree than urethanes. In some embodiments, it may be useful to enable some movement between the first antenna and patient tissues to prevent deformation of the first antenna and/or restriction of patient movement. In various embodiments, the coating disposed on the coil and/or substrate can comprise the same material as the substrate, to facilitate bonding of the coating to the substrate. In some embodiments, the substrate serves as a coating to isolate the coil from the environment and/or the substrate and/or coil do not carry any coating(s).

An electronics component of the electronics package of the present technology may be hermetically enclosed (e.g., sealed or otherwise hermetically covered). In some embodiments, the electronics component comprises a printed circuit board with discrete electronic components mounted on a substrate comprising polymeric layers secured together with adhesive. Such components can be susceptible to corrosion, degradation, and/or damage within the human body and/or may not be biocompatible. Accordingly, the electronics component can be positioned within an enclosure that isolates the electronics component from the environment and/or prevents fluid ingress into the electronics component. The enclosure can comprise one or more coatings and/or one or more housings. The enclosure can comprise multiple, sequentially applied coatings of different materials, multiple, sequentially applied coatings of a single material, and/or one or more coatings with regions including different materials. In some embodiments, the enclosure includes a coating comprising an epoxy, a coating comprising a polymer, a coating comprising a ceramic, and/or a coating comprising a metal. Any of the aforementioned coatings, whether utilized alone or in combination with additional coatings, can be configured to hermetically enclose the electronics component.

As but one example, an enclosure of the present technology can comprise a first coating comprising an epoxy, a second coating comprising multiple regions of different materials, and a third coating comprising a polymer. The second coating can comprise, for example, a first region comprising a parylene and a second region comprising a ceramic. In some embodiments, the second coating comprises multiple alternating regions of parylene and ceramic. In any case, the regions can be thin (e.g., about 1 μm to about 10 μm, etc.), flexible, and/or hermetically enclosing. In some embodiments, the first coating and/or the second coating are configured to provide a hermetic enclosure. Additionally or alternatively, the third coating can be similar to the coating of the first antenna. For example, the third coating can be soft, flexible, hydrophobic, biocompatible, and/or non-corrosive. In some embodiments, the third coating comprises the same material as the coating of the first antenna, such that a single molding process can be employed to apply the coating to the first antenna and the electronics component. Any of the aforementioned coatings can be applied in any suitable order. For example, either the first coating or the second coating can be applied over the PCB substrate and/or components carried by the substrate. Then the other of the first coating or the second coating can be applied over the coating applied to the PCB substrate and/or components. In some embodiments, the enclosure does not include the first coating, the second coating, and/or the third coating.

An enclosure carried by the electronics component can define one or more ports configured to receive an elongated member therethrough to connect the electronics component to one or more components external to the enclosure. For example, electrical conductors extending between the conductive elements of the lead and the electronics component can be configured to extend through a port in an enclosure carried by the electronics component. Ports and other openings in the enclosure can increase the risk of fluid ingress into the enclosure, so in some embodiments it may be desirable to reduce or limit the number of ports in the enclosure. In various embodiments, a port can be configured to receive multiple elongated members therein so that the enclosure contains fewer ports than elongated members passing through the enclosure. For example, all of the electrical conductors can pass through a single port. In some embodiments, multiple elongated members can be bundled together before passing through a port, for example as described with reference to cables 1551, 1553 (FIG. 15).

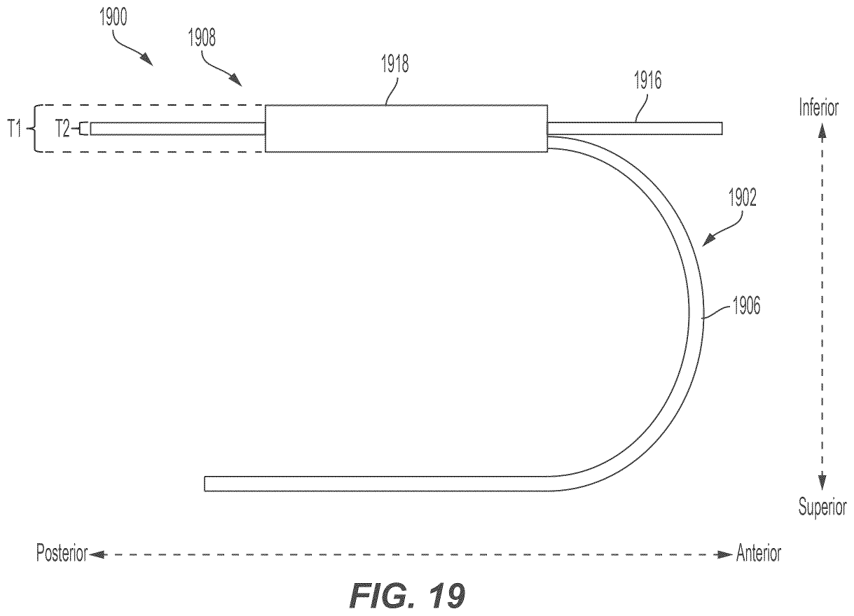

As shown in FIG. 19, in some embodiments the thickness T1 of the electronics component 1918, including its enclosure, can be larger than a thickness T2 of the first antenna 1916, including its coatings. The electronics component 1918 may have a larger thickness T1, for example, in embodiments in which the electronics component 1918 carries more coatings and/or thicker coatings than the first antenna 1916. According to various embodiments, the first antenna 1916 can be positioned along the thickness T1 of the electronics component such that the extension portion 1906 of the lead 1902 can couple to the electronics component 1918 but not the first antenna 1916. As a result, when the neuromodulation device 1900 is in an implanted configuration (as shown in FIG. 19), the extension portion 1906 can extend superiorly away from the electronics component 1918 without having to extend anteriorly along the first antenna 1916 (as compared to the neuromodulation device 100 shown in FIGS. 2A-3F, for example, which depict the extension portion 106 extending anteriorly along the first antenna 116). The lead body 1904 can extend at least posteriorly from the extension portion 1906 and may extend posteriorly beyond a posterior edge of the first antenna 1916 or may terminate anterior of the posterior edge of the first antenna 1916. In any case, coupling the extension portion 1906 to the electronics component 1918 can enable the neuromodulation device 1900 to have a maximum dimension in the sagittal plane (e.g., an anterior to posterior distance, etc.) when implanted of no greater than an expected distance between the mentum and the hyoid of a patient or a population of patients. As previously noted, the mentum and the hyoid bound the submental region anteriorly and posteriorly, respectively, and the expected mentum-hyoid distance with a neutral neck posture for a population of patients is about 35 mm to about 55 mm. The expected mentum-hyoid distance during neck flexion is about 30% to about 40% less than that of a neutral posture. Thus, the neuromodulation device 1900 can therefore have a maximum dimension in the sagittal plane when implanted of no greater than about 40 mm, no greater than about 35 mm, no greater than about 30 mm, no greater than about 25 mm, or no greater than about 20 mm. To accommodate patients with a range of mentum to hyoid distances, the neuromodulation device 1900 can have a maximum dimension in the sagittal plane when implanted of about 25 mm, about 24 mm, about 23 mm, about 22 mm, about 21 mm, about 20 mm, about 19 mm, about 18 mm, or about 17 mm.

The wound portions of the coils disclosed herein can be formed by shaping a wire into a desired pattern of turns. The wire can be manually or automatically wound to form the wound portion. As described above, in some embodiments a substrate of the first antenna includes features configured to facilitate winding and/or retention of a wire in a desired pattern of turns. For example, a human operator and/or a machine can place the wire within the grooves of the substrate to form the turns. In some embodiments, the wire can be wound into the desired pattern of turns and then inserted into the grooves of the substrate or otherwise secured to the substrate. In some embodiments, for example as described with reference to FIG. 18, inserting a wire into a lumen of a substrate comprising an elongate shaft can cause the wire to assume a desired pattern of turns. A mandrel, mold, jig, or other suitable device can be used to form the turns of the coil from the wire. The wire can be plastically deformable such that the wire retains the desired pattern of turns or the wire can be retained in the desired pattern of turns by a separate component (such as the grooves of the substrate, the lumen of the elongate shaft of the substrate, etc.). In some embodiments, the wire can be shape set while being held in the desired pattern of turns so that, after the shape setting process, the wire remains in the desired pattern of turns.

As previously noted, a first antenna with multiple coils can comprise one or more wires. A single wire can be wound to create both the turns of the coils and the electrical connectors extending between corresponding turns of the coils. In some embodiments, a single wire can be used to create the turns of one coil (e.g., two wires are used to create a first antenna with two coils, etc.). For example, a first wire can be wound into the first turns of a first coil and a second wire can be wound into the second turns of a second coil. The first and second coils can then be secured to one another via electrical connectors extending between electrical contacts of the first and second coils. In some embodiments, the first and second coils can be positioned at opposing sides of a substrate and the electrical connectors can extend through the thickness of the substrate to electrically connect corresponding turns of the first and second coils to one another. In some embodiments, the substrate can define openings with conductive material prefilled within the openings. To electrically connect the corresponding turns of the first and second coils, the first and second coils can be positioned on the opposing surfaces of the substrate and welded, heated, or otherwise electrically coupled to the conductive material within the openings at the electrical contact points along the turns. In some embodiments, a single wire can be used to create each turn of a coil (e.g., seven wires can be used to create a seven-turn coil, etc.). The individual wires can be shaped into the desired shapes of the turns, laid in a pattern corresponding to the desired pitch of the coil, and subsequently joined together.

### CONCLUSION

Although many of the embodiments are described above with respect to systems, devices, and methods for modulation of a hypoglossal nerve of a patient, the technology is applicable to other applications and/or other approaches, such as modulation of other nerves of a patient. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-19.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An implantable device comprising:
a lead comprising a proximal portion and a distal portion opposite the proximal portion along a longitudinal dimension of the lead, the distal portion comprising a first arm carrying a first electrode and a second arm carrying a second electrode, wherein the lead is configured to be implanted in a patient's body with the first arm positioned proximate a left hypoglossal nerve of the patient and the second arm positioned proximate a right hypoglossal nerve of the patient; and
an antenna positioned at the proximal portion of the lead, the antenna being configured to be positioned proximate a mylohyoid of the patient and configured to induce a current when positioned within an alternating magnetic field for supplying electrical energy to at least one of the first electrode or the second electrode, the antenna comprising a planar coil including a conductive wire formed into a wound portion with a plurality of spiral turns, and wherein the coil has a coil width measured in a first dimension and a coil length smaller than the coil width measured in a second dimension.

2. The device of claim 1, wherein the conductive wire is biocompatible and non-corrosive.

3. The device of claim 1, wherein the conductive wire comprises at least one of gold, graphene, platinum, titanium, or an alloy thereof.

4. The device of claim 1, wherein each turn extends from a first end to a second end, and wherein the wound portion defines a plane and the first and second ends of each turn lie within the plane.

5. The device of claim 1, wherein the antenna is flexible.

6. The device of claim 1, wherein the antenna comprises a substrate carrying the coil.

7. The device of claim 6, wherein the substrate defines a plurality of grooves configured to retain the plurality of spiral turns.

8. The device of claim 6, wherein the substrate is flexible.

9. The device of claim 1, wherein the antenna comprises a coating carried by the coil.

10. The device of claim 1, wherein the implantable device includes an electronics component arranged in a hermetic enclosure and electrically coupled to the antenna.

11. The device of claim 10, wherein the enclosure comprises a coating.

12. The device of claim 11, wherein the coating comprises an epoxy.

13. The device of claim 11, wherein the coating comprises a polymer.

14. The device of claim 1, wherein the planar coil is a first planar coil, the antenna further comprising a second planar coil including a second conductive wire formed into a second wound portion with a plurality of second spiral turns, wherein the second coil has a second coil width measured in the first dimension and a second coil length measured in the second dimension.

15. The device of claim 14, wherein the first and second coils are spaced apart from one another along a thickness of the antenna.

16. The device of claim 14, wherein the plurality of spiral turns of the first coil is aligned with the plurality of second spiral turns of the second coil along the coil length and coil width of the first coil and the second coil length and second coil width of the second coil.

17. The device of claim 14, wherein each turn of the first coil is individually electrically connected in parallel to a corresponding second turn of the second coil.

18. The device of claim 14, wherein the antenna comprises a substrate carrying the first and second coils.

19. The device of claim 18, wherein the substrate has a first broad side and a second broad side opposite the first broad side along a thickness of the substrate, the first coil being positioned at the first broad side and the second coil being positioned at the second broad side.

20. The device of claim 14, wherein the second conductive wire of the second coil is continuous with the conductive wire of the first coil.
